SEQUENCE LISTING (180 sequences)

SEQ ID NO: 1 - DNA, 375 bp, Human papillomavirus type 16
gcgctgccactctcagaagttattgtcaaaaacttgcaacttgctttggcaaatagctctcgaaatgctgtcgctctttctgccagccctcaactgaaagaggcccagtcagagaaggaagaagccccaaagccacttcacaaagtagtggtatgtgttagtaaaaaactcagtaagaagcagagtgaactaaatgggatcgcagcctctctaggagcagattacaggtggagttttgatgaaacagtgactcatttcatctatcaagggcggccaaatgacactaatcgggagtataaatctgtaaaagaaagaggagtacacattgtttccgagcactggcttttagattgtgcccaagagtgtaaacatctt

SEQ ID NO: 2 - protein, 125 aa, Homo sapiens
ALPLSEVIVKNLQLALANSSRNAVALSASPQLKEAQSEKEEAPKPLHKVVVCVSKKLSKKQSELNGIAASLGADYRWSFDETVTHFIYQGRPNDTNREYKSVKERGVHIVSEHWLLDCAQECKHL

SEQ ID NO: 3 - protein, 491 aa, Homo sapiens [sequence omitted]

SEQ ID NO: 4 - protein, 26 aa, Human papillomavirus type 16
RPIPKPSPWAPKKHRRLSSDQDSQTP

SEQ ID NO: 5 - protein, 8 aa, Artificial Sequence
  Description of Artificial Sequence  synthetic octapeptide antigen
MADPAAAT

SEQ ID NO: 6 - protein, 8 aa, Artificial Sequence
  Description of Artificial Sequence  synthetic octapeptide antigen
ADPAAATK

SEQ ID NO: 7 - protein, 8 aa, Artificial Sequence
  Description of Artificial Sequence  synthetic octapeptide antigen
DPAAATKY

SEQ ID NO: 8 - protein, 8 aa, Artificial Sequence
  Description of Artificial Sequence  synthetic octapeptide antigen
PAAATKYP

SEQ ID NO: 9 - protein, 8 aa, Artificial Sequence
  Description of Artificial Sequence  synthetic octapeptide antigen
AAATKYPL

SEQ ID NO: 10 - protein, 8 aa, Artificial Sequence
  Description of Artificial Sequence  synthetic octapeptide antigen
AATKYPLL

SEQ ID NO: 11 - protein, 8 aa, Artificial Sequence
  Description of Artificial Sequence  synthetic octapeptide antigen
ATKYPLLK

SEQ ID NO: 12 - protein, 8 aa, Artificial Sequence
  Description of Artificial Sequence  synthetic octapeptide antigen
TKYPLLKL

SEQ ID NO: 13 - protein, 8 aa, Artificial Sequence
  Description of Artificial Sequence  synthetic octapeptide antigen
KYPLLKLL

SEQ ID NO: 14 - protein, 8 aa, Artificial Sequence
  Description of Artificial Sequence  synthetic octapeptide antigen
YPLLKLLG

SEQ ID NO: 15 - protein, 8 aa, Artificial Sequence
  Description of Artificial Sequence  synthetic octapeptide antigen
PLLKLLGS

SEQ ID NO: 16 - protein, 8 aa, Artificial Sequence
  Description of Artificial Sequence  synthetic octapeptide antigen
LLKLLGST

SEQ ID NO: 17 - protein, 8 aa, Artificial Sequence
  Description of Artificial Sequence  synthetic octapeptide antigen
LKLLGSTW

SEQ ID NO: 18 - protein, 8 aa, Artificial Sequence
  Description of Artificial Sequence  synthetic octapeptide antigen
KLLGSTWP

SEQ ID NO: 19 - protein, 8 aa, Artificial Sequence
  Description of Artificial Sequence  synthetic octapeptide antigen
LLGSTWPT

SEQ ID NO: 20 - protein, 8 aa, Artificial Sequence
  Description of Artificial Sequence  synthetic octapeptide antigen
LGSTWPTT

SEQ ID NO: 21 - protein, 8 aa, Artificial Sequence
  Description of Artificial Sequence  synthetic octapeptide antigen
GSTWPTTP

SEQ ID NO: 22 - protein, 8 aa, Artificial Sequence
  Description of Artificial Sequence  synthetic octapeptide antigen
STWPTTPP

SEQ ID NO: 23 - protein, 8 aa, Artificial Sequence
  Description of Artificial Sequence  synthetic octapeptide antigen
TWPTTPPR

SEQ ID NO: 24 - protein, 8 aa, Artificial Sequence
  Description of Artificial Sequence  synthetic octapeptide antigen
WPTTPPRP

SEQ ID NO: 25 - protein, 8 aa, Artificial Sequence
  Description of Artificial Sequence  synthetic octapeptide antigen
PTTPPRPI

SEQ ID NO: 26 - protein, 8 aa, Artificial Sequence
  Description of Artificial Sequence  synthetic octapeptide antigen
TTPPRPIP

SEQ ID NO: 27 - protein, 8 aa, Artificial Sequence
  Description of Artificial Sequence  synthetic octapeptide antigen
TPPRPIPK

SEQ ID NO: 28 - protein, 8 aa, Artificial Sequence
  Description of Artificial Sequence  synthetic octapeptide antigen
PPRPIPKP

SEQ ID NO: 29 - protein, 8 aa, Artificial Sequence
  Description of Artificial Sequence  synthetic octapeptide antigen
PRPIPKPS

SEQ ID NO: 30 - protein, 8 aa, Artificial Sequence
  Description of Artificial Sequence  synthetic octapeptide antigen
RPIPKPSP

SEQ ID NO: 31 - protein, 8 aa, Artificial Sequence
  Description of Artificial Sequence  synthetic octapeptide antigen
PIPKPSPW

SEQ ID NO: 32 - protein, 8 aa, Artificial Sequence
  Description of Artificial Sequence  synthetic octapeptide antigen
IPKPSPWA

SEQ ID NO: 33 - protein, 8 aa, Artificial Sequence
  Description of Artificial Sequence  synthetic octapeptide antigen
PKPSPWAP

SEQ ID NO: 34 - protein, 8 aa, Artificial Sequence
  Description of Artificial Sequence  synthetic octapeptide antigen
KPSPWAPK

SEQ ID NO: 35 - protein, 8 aa, Artificial Sequence
  Description of Artificial Sequence  synthetic octapeptide antigen
PSPWAPKK

SEQ ID NO: 36 - protein, 8 aa, Artificial Sequence
  Description of Artificial Sequence  synthetic octapeptide antigen
SPWAPKKH

SEQ ID NO: 37 - protein, 8 aa, Artificial Sequence
  Description of Artificial Sequence  synthetic octapeptide antigen
PWAPKKHR

SEQ ID NO: 38 - protein, 8 aa, Artificial Sequence
  Description of Artificial Sequence  synthetic octapeptide antigen
WAPKKHRR

SEQ ID NO: 39 - protein, 8 aa, Artificial Sequence
  Description of Artificial Sequence  synthetic octapeptide antigen
APKKHRRL

SEQ ID NO: 40 - protein, 8 aa, Artificial Sequence
  Description of Artificial Sequence  synthetic octapeptide antigen
PKKHRRLS

SEQ ID NO: 41 - protein, 8 aa, Artificial Sequence
  Description of Artificial Sequence  synthetic octapeptide antigen
KKHRRLSS

SEQ ID NO: 42 - protein, 8 aa, Artificial Sequence
  Description of Artificial Sequence  synthetic octapeptide antigen
KHRRLSSD

SEQ ID NO: 43 - protein, 8 aa, Artificial Sequence
  Description of Artificial Sequence  synthetic octapeptide antigen
HRRLSSDQ

SEQ ID NO: 44 - protein, 8 aa, Artificial Sequence
  Description of Artificial Sequence  synthetic octapeptide antigen
RRLSSDQD

SEQ ID NO: 45 - protein, 8 aa, Artificial Sequence
  Description of Artificial Sequence  synthetic octapeptide antigen
RLSSDQDQ

SEQ ID NO: 46 - protein, 8 aa, Artificial Sequence
  Description of Artificial Sequence  synthetic octapeptide antigen
LSSDQDQS

SEQ ID NO: 47 - protein, 8 aa, Artificial Sequence
  Description of Artificial Sequence  synthetic octapeptide antigen
SSDQDQSQ

SEQ ID NO: 48 - protein, 8 aa, Artificial Sequence
  Description of Artificial Sequence  synthetic octapeptide antigen
SDQDQSQT

SEQ ID NO: 49 - protein, 8 aa, Artificial Sequence
  Description of Artificial Sequence  synthetic octapeptide antigen
DQDQSQTP

SEQ ID NO: 50 - protein, 8 aa, Artificial Sequence
  Description of Artificial Sequence  synthetic octapeptide antigen
QDQSQTPE

SEQ ID NO: 51 - protein, 8 aa, Artificial Sequence
  Description of Artificial Sequence  synthetic octapeptide antigen
DQSQTPET

SEQ ID NO: 52 - protein, 8 aa, Artificial Sequence
  Description of Artificial Sequence  synthetic octapeptide antigen
QSQTPETP

SEQ ID NO: 53 - protein, 8 aa, Artificial Sequence
  Description of Artificial Sequence  synthetic octapeptide antigen
SQTPETPA

SEQ ID NO: 54 - protein, 8 aa, Artificial Sequence
  Description of Artificial Sequence  synthetic octapeptide antigen
QTPETPAT

SEQ ID NO: 55 - protein, 8 aa, Artificial Sequence
  Description of Artificial Sequence  synthetic octapeptide antigen
TPETPATP

SEQ ID NO: 56 - protein, 8 aa, Artificial Sequence
  Description of Artificial Sequence  synthetic octapeptide antigen
PETPATPL

SEQ ID NO: 57 - protein, 8 aa, Artificial Sequence
  Description of Artificial Sequence  synthetic octapeptide antigen
ETPATPLS

SEQ ID NO: 58 - protein, 8 aa, Artificial Sequence
  Description of Artificial Sequence  synthetic octapeptide antigen
TPATPLSC

SEQ ID NO: 59 - protein, 8 aa, Artificial Sequence
  Description of Artificial Sequence  synthetic octapeptide antigen
PATPLSCC

SEQ ID NO: 60 - protein, 8 aa, Artificial Sequence
  Description of Artificial Sequence  synthetic octapeptide antigen
ATPLSCCT

SEQ ID NO: 61 - protein, 8 aa, Artificial Sequence
  Description of Artificial Sequence  synthetic octapeptide antigen
TPLSCCTE

SEQ ID NO: 62 - protein, 8 aa, Artificial Sequence
  Description of Artificial Sequence  synthetic octapeptide antigen
PLSCCTET

SEQ ID NO: 63 - protein, 8 aa, Artificial Sequence
  Description of Artificial Sequence  synthetic octapeptide antigen
LSCCTETQ

SEQ ID NO: 64 - protein, 8 aa, Artificial Sequence
  Description of Artificial Sequence  synthetic octapeptide antigen
SCCTETQW

SEQ ID NO: 65 - protein, 8 aa, Artificial Sequence
  Description of Artificial Sequence  synthetic octapeptide antigen
CCTETQWT

SEQ ID NO: 66 - protein, 8 aa, Artificial Sequence
  Description of Artificial Sequence  synthetic octapeptide antigen
CTETQWTV

SEQ ID NO: 67 - protein, 8 aa, Artificial Sequence
  Description of Artificial Sequence  synthetic octapeptide antigen
TETQWTVL

SEQ ID NO: 68 - protein, 8 aa, Artificial Sequence
  Description of Artificial Sequence  synthetic octapeptide antigen
ETQWTVLQ

SEQ ID NO: 69 - protein, 8 aa, Artificial Sequence
  Description of Artificial Sequence  synthetic octapeptide antigen
TQWTVLQS

SEQ ID NO: 70 - protein, 8 aa, Artificial Sequence
  Description of Artificial Sequence  synthetic octapeptide antigen
QWTVLQSS

SEQ ID NO: 71 - protein, 8 aa, Artificial Sequence
  Description of Artificial Sequence  synthetic octapeptide antigen
WTVLQSSL

SEQ ID NO: 72 - protein, 8 aa, Artificial Sequence
  Description of Artificial Sequence  synthetic octapeptide antigen
TVLQSSLH

SEQ ID NO: 73 - protein, 8 aa, Artificial Sequence
  Description of Artificial Sequence  synthetic octapeptide antigen
VLQSSLHL

SEQ ID NO: 74 - protein, 8 aa, Artificial Sequence
  Description of Artificial Sequence  synthetic octapeptide antigen
LQSSLHLT

SEQ ID NO: 75 - protein, 8 aa, Artificial Sequence
  Description of Artificial Sequence  synthetic octapeptide antigen
QSSLHLTA

SEQ ID NO: 76 - protein, 8 aa, Artificial Sequence
  Description of Artificial Sequence  synthetic octapeptide antigen
SSLHLTAH

SEQ ID NO: 77 - protein, 8 aa, Artificial Sequence
  Description of Artificial Sequence  synthetic octapeptide antigen
SLHLTAHT

SEQ ID NO: 78 - protein, 8 aa, Artificial Sequence
  Description of Artificial Sequence  synthetic octapeptide antigen
HLTAHTKD

SEQ ID NO: 79 - protein, 8 aa, Artificial Sequence
  Description of Artificial Sequence  synthetic octapeptide antigen
HLTAHTKD

SEQ ID NO: 80 - protein, 8 aa, Artificial Sequence
  Description of Artificial Sequence  synthetic octapeptide antigen
LTAHTKDG

SEQ ID NO: 81 - protein, 8 aa, Artificial Sequence
  Description of Artificial Sequence  synthetic octapeptide antigen
TAHTKDGL

SEQ ID NO: 82 - protein, 8 aa, Artificial Sequence
  Description of Artificial Sequence  synthetic octapeptide antigen
AHTKDGLT

SEQ ID NO: 83 - protein, 8 aa, Artificial Sequence
  Description of Artificial Sequence  synthetic octapeptide antigen
HTKDGLTV

SEQ ID NO: 84 - protein, 8 aa, Artificial Sequence
  Description of Artificial Sequence  synthetic octapeptide antigen
TKDGLTVI

SEQ ID NO: 85 - protein, 8 aa, Artificial Sequence
  Description of Artificial Sequence  synthetic octapeptide antigen
KDGLTVIV

SEQ ID NO: 86 - protein, 8 aa, Artificial Sequence
  Description of Artificial Sequence synthetic octapeptide antigen
DGLTVIVT

SEQ ID NO: 87 - protein, 8 aa, Artificial Sequence
  Description of Artificial Sequence synthetic octapeptide antigen
GLTVIVTL

SEQ ID NO: 88 - protein, 8 aa, Artificial Sequence
  Description of Artificial Sequence synthetic octapeptide antigen
LTVIVTLH

SEQ ID NO: 89 - protein, 8 aa, Artificial Sequence
  Description of Artificial Sequence synthetic octapeptide antigen
TVIVTLHP

SEQ ID NO: 90 - protein, 92 aa, Human papillomavirus type 16
MADPAAATKYPLLKLLGSTWPTTPPRPIPKPSPWAPKKHRRLSSDQDQSQTPETPATPLSCCTETQWTVLQSSLHLTAHTKDGLTVIVTLHP

SEQ ID NO: 91 - protein, 126 aa, Human papillomavirus
MADNKAPQGLLGLLQYTPTTQPYPRVTPPSNRRPSTTPNSQDRGRPRRSDKDSRKHLYADGLTDGEDPEVPEVEDEEKENQRPLGHPDLSLLRETLEVYTQRLKRDILQQDLDDFCRKLGIHPWSV

SEQ ID NO: 92 - protein, 117 aa, Artificial Sequence
  Description of Artificial SequenceHPV E4 Consensus Amino Acid Sequence
MADAPTQYPLLKLLPPTPTPPPRPPPPPPPPRPWAGPKKPTRGPPRRRRLESDSDDSGEVEPTPTTPPAPPTGDEEVEPPWTVQTLLSSVTLTWEYTFDGLVVIVQDLEDYWKKLHL

SEQ ID NO: 93 - protein, 49 aa, Artificial Sequence
  Description of Artificial Sequence HPV E4 protein consenus amino acid sequence, amino acids 27-73.
PPPPPRPWAGPKKPTRGPPRRRRLESDSDSDSGEVEGPTPTTPPAPPTG

SEQ ID NO: 94 - protein, 35 aa, Human papillomavirus type 54
HHVPTTPQKQSRARRRLENELESTAQTSNHTAPQT

SEQ ID NO: 95 - protein, 48 aa, Human papillomavirus type 32
PSQGVTATTAQTEYYTKTPPRPPRRENDTDSLCSHQQSTCSTTASQTY

SEQ ID NO: 96 - protein, 50 aa, Human papillomavirus type 42
PLTTTTQTVQTEQHTTCPSKPHRHENDTDSVDSRHHSTCSTQTPASPASP

SEQ ID NO: 97 - protein, 47 aa, Human papillomavirus type 3
KPRWARPKDRSKSDSDSRRSTGSSSSNSSSNSNSNNIPKPPPRKPLN

SEQ ID NO: 98 - protein, 39 aa, Human papillomavirus type 28
KPRWARPKDRSKNDSDSRHSTGSSSSDSTPKPPPRKPLN

SEQ ID NO: 99 - protein, 40 aa, Human papillomavirus type 10
KPRWARPRDRNKSDSDSRRSTDSTSSSDKGPKIPPRRPRN

SEQ ID NO: 100 - protein, 51 aa, Human papillomavirus type 29
KPRWGLRRDRNGNDAGLKQSGLGHSSSSSSSTSSSSSNRPRPTPPPRKPVH

SEQ ID NO: 101 - protein, 41 aa, Human papillomavirus type 61
PPRAWAPPRHPPRCRRRLISDSDSTETESSSPTQHKKTTTS

SEQ ID NO: 102 - protein, 45 aa, Human papillomavirus type 2a
QEEQLRPPKRCAPPRRQRVRRPSASVSSSDSSIPGPTLRERSERG

SEQ ID NO: 103 - protein, 45 aa, Human papillomavirus type 27
EQEQLRPQTCCAPPRRHRVRRPSASGSSSDSSISGPTLRERSERG

SEQ ID NO: 104 - protein, 41 aa, Human papillomavirus type 57
QSRPHSRTPPRRHRVRHPSASGSSSDSSGNSPTLRGRSEKG

SEQ ID NO: 105 - protein, 39 aa, Human papillomavirus type 26
PTCPWAPRKPRRHTQESDDDSVDLTPPSPQSPLSPQLPH

SEQ ID NO: 106 - protein, 40 aa, Human papillomavirus type 30
PPRPWAPTKPRPPHGRENVLEPQSPTVQTPPDSPLPESPT

SEQ ID NO: 107 - protein, 40 aa, Human papillomavirus type 53
PPRPWAPTKPHHPCGRENVPEPQSPTVLTPPHSPLPQPES

SEQ ID NO: 108 - protein, 39 aa, Human papillomavirus type 56
PPPRPWATKTPQYPTDQENDPDYGNQNLTPPESPTQSVS

SEQ ID NO: 109 - protein, 37 aa, Human papillomavirus type 66
PPLWAPKTPRYPTDQENDPEQVNQNLTPPESPTHTVS

SEQ ID NO: 110 - protein, 29 aa, Human papillomavirus type 18
APCPWAPQRPTARRRLLHDLDTVDSRRSS

SEQ ID NO: 111 - protein, 31 aa, Human papillomavirus type 45
KPHPWAPQNPTSRRRLLSDLDSVDSQSSTTD

SEQ ID NO: 112 - protein, 32 aa, Human papillomavirus type 39
PQQPHAPKKQSRRRLESDLDSVQSQSPLSPTE

SEQ ID NO: 113 - protein, 27 aa, Human papillomavirus type 70
PQQPHAPKKLSRRRLASVESPDPQKQT

SEQ ID NO: 114 - protein, 32 aa, Human papillomavirus type 59
KPRTWAPKRGTVRRRLESDQDSVDTHSTLSLP

SEQ ID NO: 115 - protein, 46 aa, Human papillomavirus type 7
PPTPPRCTTPPTPCPRRPPKYTTTATHRPESEGETETCPSVQWTDV

SEQ ID NO: 116 - protein, 41 aa, Human papillomavirus type 40
PTPPTPPPQRPPKRSAPPRHRPESDEETDTCPSPLLWANHS

SEQ ID NO: 117 - protein, 35 aa, Human papillomavirus type 16
KPSPWAPKKHRRLSSDQDQSQTPETPATPLSCCTE

SEQ ID NO: 118 - protein, 34 aa, Human papillomavirus type 35
KPAPWAPQKPRRQITNDFEGVPSSPTTPPSECDS

SEQ ID NO: 119 - protein, 39 aa, Human papillomavirus type 31
KPAPWAPVKVCGGRRRLLSDQEQSQSTETPTTPTSCCEA

SEQ ID NO: 120 - protein, 38 aa, Human papillomavirus type 52
PQCPWVPKTHTYNHHRNDDDQTSQTPETPSTPTTFCGD

SEQ ID NO: 121 - protein, 26 aa, Human papillomavirus type 33
HHKQRPNDDDLQTPQTPPSPLQSCSV

SEQ ID NO: 122 - protein, 32 aa, Human papillomavirus type 58
PTTKVHRGQSDDDSIYQTPETTPSTPQSIQTA

SEQ ID NO: 123 - protein, 29 aa, rhesus monkey papillomavirus
PTPAPRKTCGHRLQSECVGQTQVEIQCGP

SEQ ID NO: 124 - protein, 36 aa, Human papillomavirus type 66
PLCPQAPRKTQCKRRLGNEHEESNSPLATPCVWPTL

SEQ ID NO: 125 - protein, 34 aa, Human papillomavirus type 11
LQCPPAPRKTACRRRLGSEHVDRPLTTPCVWPTS

SEQ ID NO: 126 - protein, 40 aa, Human papillomavirus type 44
HRPHPHCPLAPPRTAWTRRHVNDPEDPPQTPTTPETPSVS

SEQ ID NO: 127 - protein, 40 aa, Human papillomavirus type 55
HRPHLHCPPAPPRNAWTRRHVNDPEDPPQTPTTPGTPSVS

SEQ ID NO: 128 - protein, 39 aa, Human papillomavirus type 13
PQCPAAPRKNVCKRRLVNDNEDLHVPLETPRTHKALCVS

SEQ ID NO: 129 - protein, 40 aa, Pygmy chimpanzee papillomavirus
AQCHPSPQKIVCKKRRPINDFEDPPTVLENSKTPLTLCVP

SEQ ID NO: 130 - protein, 22 aa, Human papillomavirus type 34
ATHRTRVCQHGNGIDSVTQTRG

SEQ ID NO: 131 - protein, 71 aa, Human papillomavirus type 19
GTRDDLPAGPDDKPKRARNDQGPNPSPGRGRGRGLFRLTGDHDPNPEERPPPLEGEVEGHPPPPVTNPPGH

SEQ ID NO: 132 - protein, 95 aa, Human papillomavirus type 25
PPAGHDDSKPKRARGDQGPSPGPGPSPAPVSDRGRGRGRGLNLSRLSGDQDPDPEEKPQPEGEVQGHPQPPPVTEPQGHLPPPPLPPPNGHNDRD

SEQ ID NO: 133 - protein, 99 aa, Human papillomavirus type 20
GTDGDLPVGQGEQPKRARGDGPGQSPSPSPGRGRGRGTGLGLGLGLNRRAGGLGTDHDPDPEGESPSAPLPPPPQPPPDGQVEGHPPPPPPPPHNGRDS

SEQ ID NO: 134 - protein, 106 aa, Human papillomavirus type 21
GTDGDRPVGPGERPKRIKGGDRGPSPGRGRGRGRGSDPDPGPDPGPIPGPGLNRLTSRNTDSDPEGKCPSSLPPPPPPPPQPTTPPEGQGEGHPPPPPPPPNGHDG

SEQ ID NO: 135 - protein, 88 aa, Human papillomavirus type 14
EGTDADRPVGPGERPKRGRGGDRGPSPGRGRGRGLGSDLDPGRNRLSGGLGTDQDPDPDKKKCPESQPPPEGEVEGHPPPPPNGHNGH

SEQ ID NO: 136 - protein, 90 aa, Human papillomavirus type 5
SQGDRKRSKGDQGPDTGPGLGPGRGPSPKPTPLGPPPGPGPRRSPRLGPLQADRDPEEGPQPPAEGEVEGHPGGDQGHPPPPPPAPHNGH

SEQ ID NO: 137 - protein, 87 aa, Human papillomavirus type 36
GQGDRKRSKGDQGPDTDPLGPDRGPSPGPTPQPLGLPPPGLGPRRSPRLGSSGYQPDHDPEAPLEGEVEGGGHGHHPPPPPPPPPTN

SEQ ID NO: 138 - protein, 87 aa, Human papillomavirus type 47
QGDRKRTKGDPDPDPGRGPVLKPTLPPPPPPPPTGPGLRRSTRLVLVPGQGPPPDLPAPPVEGEVEGHPQGKDRDHPPPTPQNGHGK

SEQ ID NO: 139 - protein, 76 aa, Human papillomavirus type 12
GDRKRSKGDQGRDTAPSLTPGRAPSPKPGPLAPPPYPGPPGPRRSHRLGTGGRDRNPEEGGVEGHPPTPPLSGGDP

SEQ ID NO: 140 - protein, 91 aa, Human papillomavirus type 8
QDRKKSRGDQGRDTAPGLAPGRSPGLGPLAPPPYPGPGPRRSPRQFGPGPDRDPEDGLQPPLGEGQVEGHPGDGDQPQGHPPPTPSNGHKG

SEQ ID NO: 141 - protein, 70 aa, Human papillomavirus type 24
PLTPDADDDPRPGKRSKGDEHGPAPGRAAAPLKLDLDPPQGGPDQPPGATGGVGETPPEGNEESQPPPGE

SEQ ID NO: 142 - protein, 61 aa, Human papillomavirus type 15
TTEKNLAQPPPPGGRKDKDKDKKTQQGDQGPPQGGDKKSPGEGTSADGDDPEKPPSPPPGE

SEQ ID NO: 143 - protein, 60 aa, Human papillomavirus type 17
DTGGKRLALQPPPPGTKDKTSDDQGPPHGGDKQSPGEGSDASGDENAPTPETPQDPPTGE

SEQ ID NO: 144 - protein, 62 aa, Human papillomavirus type 37
EEKHLALQPPPPGKKDKEKTPQQGDQGPPPGGNKQPPGEGTDADGDENAPTPETPPVPPTGE

SEQ ID NO: 145 - protein, 61 aa, Human papillomavirus type 9
PPPGRKDRDKEKEKEKEKEKKPTTGDKGPDPRVEQKPKGEGSDGDEEGPPPQTPLPPPTGE

SEQ ID NO: 146 - protein, 57 aa, Human papillomavirus type 22
LVLQSPPSGGKKGERDKDKKPQQGEEKPDQGPEAPSSGEGGPPDDPSPENPQNPPGG

SEQ ID NO: 147 - protein, 55 aa, Human papillomavirus type 23
KHLALQPPPGGKKDKEKKPSPGEEKPDQGPGAESNGGGGKPKDPPPEEPQNPPGG

SEQ ID NO: 148 - protein, 63 aa, Human papillomavirus type 38
DTGEKHLALQPPPAGKGKDKEKPQAPKGEEKADQGPEAPTGEGGTPGDPPPEDPQSPPPGEGE

SEQ ID NO: 149 - protein, 75 aa, Human papillomavirus type 49
TLVLQQPPTPGKRSRDDDPGLEPGPADGKRAPQGPKKPAVPDPDPDPLPEDPEGPEDLSQPPEIPAPREPAGAEG

SEQ ID NO: 150 - protein, 52 aa, Human papillomavirus type 4
PSRRALLEGGNRGNPTRPPPRPLKPREYDYDEDDEKENQGPGQEKPPAKEEE

SEQ ID NO: 151 - protein, 54 aa, Human papillomavirus type 65
PSLPRRALVVGGNRGNLNRPPQRPPKPRGYEYDEDDDKENQGPGQERPPAKEEE

SEQ ID NO: 152 - protein, 44 aa, Human papillomavirus type 48
LEGDRASQKTPTPSRPPPRHPDYESDDDENRENLEPPTPPHPED

SEQ ID NO: 153 - protein, 48 aa, Human papillomavirus type 50
ANRKDLEAVNQKPYRTPNHPPRHQQYDFDEDDEKENTIPTDTESHNQN

SEQ ID NO: 154 - protein, 47 aa, Human papillomavirus type 60
LPTEDRPHKRESLALPRRRVLFDYDAEDPTSNKENYPPESRPVPKDA

SEQ ID NO: 155 - protein, 39 aa, Bovine papillomavirus type 1
PSLSLLCSAPPPAVPSEQASVGYETVLARTPTIFLQARG

SEQ ID NO: 156 - protein, 39 aa, Unknown
  Description of Unknown Organism Non-Human Virus
PSLSLLCSAPPPAYPSEQASVGYETVLARTPTIFLQARG

SEQ ID NO: 157 - protein, 43 aa, European elk papillomavirus
PTQPTEPCLTLLLDNPPFVAPSELAKTGVGPFTARLPTAHHHP

SEQ ID NO: 158 - protein, 34 aa, deer papillomavirus
TLLLEATPFTVPSELAKTGVGPLTARLPTAHHSP

SEQ ID NO: 159 - protein, 50 aa, Bovine papillomavirus type 4
PDLPETPGAGSRGRSRLRDRDHGHDHDRLRRGRTPVDETRGYRVPGDPRE

SEQ ID NO: 160 - protein, 50 aa, Human papillomavirus type 41
PQRYYDRRGRDDAETRKRGSRSPQPLSEDEELTDADPPRRPNAGPRRRLF

SEQ ID NO: 161 - protein, 47 aa, Canine oral papillomavirus
LPPGKGRHGGLDGGRRGSPEGQEDEEDSDEEEAENYPPSRSRPRRGR

SEQ ID NO: 162 - protein, 34 aa, Unknown Organism
  Description of Unknown Organism cottontail rabbit papillomavirus
QGPKPRVHWADEGQGHQGCNEGRQSNENRPPRTK

SEQ ID NO: 163 - protein, 61 aa, Unknown Organism
  Description of Unknown Organism Rabbit Oral Papillomavirus
LQYPQAPRTIRKPRSSRYRGRFLVTDGGDPDPQELDSTQQDPEDKENIPPTSTPTPSPPTP

SEQ ID NO: 164 - protein, 48 aa, Human papillomavirus type 1a
TPPSNRRPSTTPNSQDRGRPRRSDKDSRKHLYADGLTDGEDPEVPEVE

SEQ ID NO: 165 - protein, 40 aa, Human papillomavirus type 63
KLPEKQRRRGRDTTRNRRLFASDGPTDEEGPEVPEIPPSD

SEQ ID NO: 166 - protein, 48 aa, Mastomys natalensis
IPRVSLQDKTTGGNQQRRRRRGERGARTPSPETTAQRPKRPRRACTRK

SEQ ID NO: 167 - protein, 15 aa, Human papillomavirus type 16
RRIPKPSPWAPKKHR

SEQ ID NO: 168 - protein, 12 aa, Human papillomavirus type 16
PKPSPWAPKKHR

SEQ ID NO: 169 - protein, 13 aa, Artificial Sequence
  Description of Artificial Sequence synthetic peptide
MADPAAATKYPLC

SEQ ID NO: 170 - protein, 8 aa, Artificial Sequence
  Description of Artificial Sequence Synthetic Peptide
LLRGAFDY

SEQ ID NO: 171 - protein, 11 aa, Artificial Sequence
  Description of Artificial Sequence Synthetic Peptide
NSRDSSGGNAV

SEQ ID NO: 172 - protein, 8 aa, Artificial Sequence
  Description of Artificial Sequence synthetic peptide
LVQGSFDY

SEQ ID NO: 173 - protein, 8 aa, Artificial Sequence
  Description of Artificial Sequence synthetic peptide
QADSSTHV

SEQ ID NO: 174 - DNA, 72 bp, Artificial Sequence
  Description of Artificial Sequence  synthetic oligonucleotide
cctgttgtgagcctcctgtcgaannnnnnnnnnnnnnnnnnnnnnnnnnttgagcgtttattcttgtctccc

SEQ ID NO: 175 - DNA, 40 bp, Artificial Sequence
  Description of Artificial Sequence  synthetic oligonucleotide
taatacgactcactatagggagacaagaataaacgctcaa

SEQ ID NO: 176 - DNA, 24 bp, Artificial Sequence
  Description of Artificial Sequence  synthetic oligonucleotide
gcctgttgtgagcctcctgtcgaa

SEQ ID NO: 177 - DNA, 90 bp, Artificial Sequence
  Description of Artificial Sequence synthetic oligonucleotide, n = any randon nucleotide.
taatacgactcactatagggagacaagaataaacgctcaannnnnnnnnnnnnnnnnnnnnnnnnnttcgacaggaggctcacaacaggc

SEQ ID NO: 178 - RNA, 73 nt, Artificial Sequence
  Description of Artificial Sequencein vitro transcription product from SEQ ID NO 177, n = any random nucleotide.
gggagacaagaauaaacgcucaannnnnnnnnnnnnnnnnnnnnnnnnnuucgacaggaggcucacaacaggc

SEQ ID NO: 179 - protein, 4 aa, Artificial Sequence
  Description of Artificial Sequence synthetic peptide
DEAD

SEQ ID NO: 180 - protein, 18 aa, Human papillomavirus type 51
  misc_feature (18)..(18): Xaa can be any naturally occurring amino acid
LPPAWAPKKPRHNSENDX

(19) United States
(12) Patent Application Publication
(10) Pub. No.: US 2003/0219726 A1
(43) Pub. Date: Nov. 27, 2003

(54) Detection of abnormalities leading to cervical malignancy

(75) Inventor: John Doorbar, London (GB)

Correspondence Address: PALMER & DODGE, LLP, KATHLEEN M. WILLIAMS, 111 HUNTINGTON AVENUE, BOSTON, MA, 02199, US

(73) Assignee: Medical Research Council

(21) Appl. No.: 10/350,719

(22) Filed: Jan. 24, 2003

Related U.S. Application Data
(63) Continuation of application No. PCT/GB01/01176, filed on Mar. 16, 2001.

(30) Foreign Application Priority Data
Jul. 24, 2000 (GB) 0018140.4

(51) Int. Cl. C12Q 1/70; C12Q 1/68

(52) U.S. Cl. 435/5; 435/6

(57) ABSTRACT

The invention relates to a method of screening for abnormalities which are indicative of, or can lead to, cervical malignancy. The invention also relates to a method of screening for human papillomavirus (HPV) infections and precursor lesions and other conditions which precede or concur with cervical malignancy, and reagents and kits for use in these methods.

### FIELD OF THE INVENTION

This invention relates to a method of simultaneously screening for abnormalities which are indicative of, or can lead to, cervical malignancy. The invention therefore relates to a method of simultaneously screening for HPV infections and precursor lesions or other conditions which precede or concur with cervical malignancy, and to reagents of use in the above methods.

### BACKGROUND OF THE INVENTION

Despite an intensive and expensive national screening programme, carcinoma of the cervix is the eighth most common malignancy of women in the UK and the most common malignancy in women under 35 years of age (Cancer Research Campaign, Cancer of the cervix uteri. 1994, CRC: London). In the developing world it is the most common malignancy and the leading cause of death in women between the ages of 35-45 years, with an estimated 437,000 new cases each year (Cancer Research Campaign, Cancer—world perspectives. 1995, CRC: London

Papillomaviruses (PVs) cause epithelial tumours in humans which vary in severity depending on the site of infection and the HPV (human papilloma virus) type involved (Laimins, 1993; Villiers de, 1994). Low risk types such as HPV 1 or HPV63 (Egawa et al, 1993a; Egawa et al, 1993b) cause benign cutaneous warts which progress to malignancy only rarely, while high risk viruses such as HPV16 and HPV31 cause flat warts at mucosal sites, and are associated with high grade cervical intraepithelial neoplasia (CIN) and cancer (Schneider, 1994). Formation of an HPV-induced tumour is thought to require infection of an epithelial basal cell, and the expression of viral early proteins in order to stimulate cell proliferation. The late stages of the virus life cycle, which ultimately lead to the production of infectious virions, are initiated only as the infected cell migrates through the upper differentiated layers of the epidermis.

It is well known that there is a very strong correlation between HPV-infection and development of cervical carcinoma: over 99% of women (Walboomers et al., J Pathol 189:12-19) with cervical carcinoma show evidence of HPV infections of the cervix. Accordingly, one possible alternative to conventional histopathological examination of cervical smears is to examine samples for evidence of HPV infection. For example, there have been numerous proposals to screen for cervical carcinoma by performing DNA hybridisation assays on samples, using nucleic acid probes specific for HPV sequences. More recently, methods of screening for evidence of HPV infections by analysing the expression of HPV polypeptides have been described, for example in WO98/25145.

In an unconnected approach, it has been possible to detect cellular abnormalities using molecules directed against particular proteins of the preinitiation complex of DNA replication. Especially useful are binding molecules directed against Cdc6, and those directed against MCM proteins, such as MCM5. These are described in WO 99/21014. Binding molecules directed against Cdc6, and also those against MCM2, MCM3, MCM4, MCM5, MCM 6 or MCM7 are effective in marking cellular growth abnormality, as are antibodies against PCNA and Ki67 or Ki5134 (for example see Southern, S. A. & Herrington, C. S. (1998) Cancer Research 58, 2941-2945 or Follen Mitchell, M. et al., (1996) Journal of the National Cancer Institute Monographs 21, 17-25). All these proteins can be considered “proliferation markers”. Of particular interest in the context of screening is the assessment of cervical samples using anti-Cdc6 or anti-MCM binding molecules which show a high-level staining of abnormal cells. This approach detects abnormalities at a much later stage, once the cells are already proliferating in the patient.

In many countries there are screening programmes to detect the presence of cervical carcinoma at an early stage. Generally such programmes operate by obtaining cervical smears from women potentially at risk of developing cervical cancer, with the resulting smears routinely being examined by conventional histopathological techniques. These techniques are laborious and time-consuming, require considerable experience to interpret results correctly, and frequently give rise to relatively large percentages of false positive results, causing unnecessary alarm. False negatives can occur when screening is carried out by inexperienced personnel and can lead to the classification of pre-cancerous lesions or other abnormalities as normal.

Both the viral polypeptide-based method and the method based on screening for markers of cell division suffer from false positive and false negative results. There is thus a need for an improved cervical cancer screening method.

### SUMMARY OF THE INVENTION

It has surprisingly been determined that a combination of two prior art techniques, based on screening for viral infection and for markers of cell division, reduces the incidence of false results in cervical screening. A single test based on both principles exploits synergy between the two detection systems to provide a streamlined and efficient test with minimal false results. In addition, the test provides for means by which the severity of a cervical lesion may be assessed and stratified.

Despite the association between HPV infection and the development of cervical cancer, the viral gene products involved in cell proliferation such as E6 and E7, are not generally regarded as useful markers of cervical neoplasia due to their low levels of expression. The E4 and L1 gene products are by contrast expressed at very high levels, and can easily be detected in the surface layers of CIN (Doorbar, J., Foo, C., Coleman, N., Medcalf, E., Hartley, O., Prospero, T., Napthine, S., Sterling, J., Winter, G. & Griffin, H. (1997) Virology 238, 40-52). E4 expression marks the onset of vegetative viral genome amplification, while the appearance of L1 in a subset of the E4-positive cells marks the start of virus synthesis.

The invention provides for the identification of cells expressing the E7 gene product by staining for E2F regulated cellular proteins such as PCNA, cyclin A (DeGregori, J., Kowalik, T. & Nevins, J. R. (1995) Mol Cell Biol 15, 4215-4224) and MCM5 (Ohtani, K., Iwanaga, R., Nakamura, M., Ikeda, M., Yabuta, N., Tsuruga, H. & Nojima, H. (1999) Oncogene 18, 2299-2309) that are induced by E7 (Huang, P. S., Patrick, D. R., Edwards, G., Goodhart, P. J., Huber, H. E., Miles, L., Garsky, V. M., Oliff, A. & Heimbrook, D. C. (1993) Mol Cell Biol 13, 953-960; Zerfass, K., Schulze, A., Spitkovsky, D., Friedman, V., Henglein, B. & Jansen-Durr, P. (1995) J Virol 69, 6389-6399). In uninfected cervix, the expression of such proteins is restricted to the dividing basal cells, where as in cervical neoplasia their expression extends into higher layers as a result of E7 expression. By combining the detection of surrogate markers of E7 activity with the detection the HPV E4 and L1 proteins it has been possible to rapidly distinguish the three major phases of the virus life cycle in high and low grade cervical neoplasia. In condyloma and in all the productive infections which have been examined, cellular proliferation, viral genome amplification and virus synthesis follow a highly ordered pattern with only E4 and E4/L1-expressing cells persisting into the surface layers. In abortive infections such as HSIL however, the life cycle of the virus is incomplete and cells expressing surrogate markers of E7 activity can persist into the surface layers. The expression patterns of these three markers in the surface layers of cervical neoplasia reflects the extent to which the virus life cycle is disturbed. This correlates precisely with the grade of cervical neoplasia in the underlying epidermis. The application of such markers allows a more accurate assessment of cervical disease.

In accordance with the present invention, it has now been demonstrated that abnormalities can be detected in a sample taken from a patient by using molecules which bind to HPV polynucleotides or polypeptides, and molecules which bind to members of the pre-initiation DNA complex or to cellular markers of viral activity. In particular, the invention provides a method of screening samples for cervical lesions, using molecules which bind to HPV polynucleotides or polypeptides, and molecules which bind to members of the pre-initiation DNA complex or to cellular markers of viral activity.

In a first aspect, the invention provides a method for detecting abnormalities in a sample from a patient, said method comprising obtaining a sample of the patient's cells, contacting said cells with two or more molecule(s), wherein at least one molecule is capable of binding a papilloma virus associated nucleic acid or antigen, and at least one molecule is capable of binding a cell proliferation marker or a marker of viral activity, and monitoring said binding.

Preferably, the cell proliferation marker or marker of viral activity is induced by E6 and/or E7.

Preferably, the cells are intact. However, cell lysates, homogenates or otherwise disrupted cells may be used as a substrate for the present invention and included in the definition of the term “cells”.

An abnormality may be a lesion, such as a pre-cancerous lesion resulting from a mucosal papilloma virus infection in an organism, or may be abnormally proliferating cells whether malignant or otherwise.

The term ‘molecule’ is used to refer to any molecule having the desired binding properties referred to above, and in more detail below. Advantageously, the molecule may be an antibody, or an antigen binding fragment thereof.

The binding of the molecule may be assessed by any suitable means, such as by use of a fluorescent label, a radioactive label, an enzymatic label, or any other label, or may be assessed by any other suitable method(s) such as surface plasmon resonance. Preferably, binding of the molecule is assessed by fluorescent labelling.

A “sample” means a suitable quantity of cervical material, in particular cervical cells. The sample could take the form of a biopsy, cells from a swab, a smear or other means of obtaining cervical cells. Preferably, the sample comprises cervical cells. More preferably, the sample comprises cells collected from a cervical smear.

“Simultaneously” means substantially at the same time. In this context, it may mean within the same experimental testing procedure, which may itself span many hours. Two tests will be considered to be performed simultaneously if they are carried out as part of one continuous string of operations carried out on the whole or part of the same sample. It is a preferred embodiment of the present invention that the several tests described are performed on the sample simultaneously.

Molecules binding to human papilloma virus polynucleotides or polypeptides may also bind to, or have been raised against, other papilloma virus polynucleotides or polypeptides. The papilloma virus referred to herein may include other papilloma type viruses. Preferably, the virus is human papilloma virus. More preferably, the virus is human papilloma virus and comprises one or more types selected from the group consisting of HPV types 16, 18, 33, 35, 45, 51, 52, 56, 58 and 61. Molecules according to the invention may bind to numerous viral polynucleotides or polypeptides.

Advantageously, the molecules according to the invention bind to HPV polypeptides. HPV DNA may be found in a proportion of cervical smears with no apparent viral infection or pathology. Hence, the use of molecules binding to HPV polypeptides provides a superior assay to tests for HPV nucleic acid. Preferably, at least one molecule is a molecule that binds specifically to a subset of HPV E4 proteins. More preferably at least one molecule is a molecule capable of binding to the papilloma virus-E4 protein, and is capable of binding within a hydrophilic region of the E4 sequence.

The invention preferably involves the detection of HPV proteins and markers of cell proliferation, or proteins that are expressed in the cell as a result of the presence of viral gene products such as p16 (see Sano, T. et al (1998) Am. J. Pathol. 153, 1741-1748; Sano, T., et al (1998) Pathol. Int. 48, 580-585; Lukas, J., et al (1994) J Cell Biol 125, 625-638.). Molecules binding to cell proliferation markers may advantageously be used in the methods of the present invention. Cell proliferation markers are gene products which are expressed in actively dividing cells, or cells which are committed to or are entering the cell cycle. These markers are generally absent from cells which are quiescent, dormant, in stationary phase or otherwise arrested either temporarily or permanently and are not participating in the cell cycle. Preferably cell proliferation markers according to the present invention comprise one or more polypeptides which are members of a preinitiation complex of DNA replication. More preferably the proliferation marker comprises one or more polypeptides selected from the group consisting of CDC6, MCM2, MCM3, MCM4, MCM5, MCM6, MCM7, Cdc7 protein kinase, Dbf4, Cdc14 protein phosphatase, cyclin A, Cdc45, PCNA, Ki67, KiS1, and MCM10. Cell proliferation markers may be detected at the mRNA stage. Preferably cell proliferation markers are detected as polypeptides.

In a preferred aspect, the cell proliferation marker is not an MCM molecule. Advantageously, it is PCNA, Ki67 and cyclin A.

The cell proliferation marker may moreover be replaced with a marker for viral activity, such as cyclin B or p16.

Molecules suitable for use in the methods of the present invention may bind to more than one target polypeptide. The invention accordingly provides a method for detecting abnormalities in a sample from a patient, said method comprising; obtaining a sample of the patient's cells; contacting said cells with two or more molecule(s), wherein at least one molecule is capable of binding a papilloma virus associated nucleic acid or antigen or a plurality of papilloma virus associated nucleic acid or antigens, and at least one molecule is capable of binding a cell proliferation marker or a plurality of cell proliferation markers, and monitoring said binding.

Reagents described and referred to herein may be advantageously supplied with instructions for the working of the invention in kit form. The present invention accordingly provides a kit comprising two or more molecule(s), wherein at least one molecule is capable of binding a papilloma virus associated nucleic acid or antigen, and at least one molecule is capable of binding a cell proliferation marker, and instructions for use of said molecules in a method described herein.

The invention moreover provides a molecule is capable of binding a papilloma virus associated nucleic acid or antigen, and a molecule is capable of binding a cell proliferation marker, for simultaneous, simultaneous separate or sequential use in the detection of abnormalities in a sample.

The ability of the invention to assess two separate indicators of cellular abnormality which can lead to cervical tumours permits the categorisation of lesions according to the severity of the risk posed to the patient, and therefore allow a clinician to prioritise urgent treatment in more serious cases.

A pattern of events necessary for completion of the virus life cycle is presented herein. In the lower layers of all productive infections including those caused by HPV1, 2 and 11, staining for E2F-activated genes such as PCNA or MCM is widespread. The extent to which these cells extend into the upper layers varies depending on the infecting HPV type, but in productive infection such proteins are never found in the surface layers. E4 expression begins in these replication competent cells as might have been predicted from previous studies showing that E4 expression coincides with the onset of genome amplification (Doorbar, J., Foo, C., Coleman, N., Medcalf, E., Hartley, O., Prospero, T., Napthine, S., Sterling, J., Winter, G. & Griffin, H. (1997) Virology 238, 40-52). Results obtained with different HPV types confirm the close association between vegetative viral DNA replication and E4 expression. The eventual loss of PCNA and MCM staining in these E4-positive cells marks completion of viral DNA replication and that packaging of the HPV genomes into infectious virions can occur. The molecular events which regulate this final stage of the virus life cycle are poorly understood, but it is likely that packaging is linked to the completion of genome amplification as it is in other DNA viruses (Wiley, S. R., Kraus, R. J., Zuo, F. G., Murray, E. E., Loritz, K. & Mertz, J. E. (1993) Genes Dev. 7, 2206-2219). The most striking observation to arise from the present work is that every cell supporting productive infection will expresses one or more of these proteins. The particular combination of proteins present in any particular cell identifies a particular stage in the virus life cycle.

The present invention demonstrates that the histological status of the cervix can be predicted from the expression pattern of just three proteins (E4\hat{ }L1 and a marker of proliferation or viral infection) in cells in the upper epithelial layers. The different combinations of these three markers in cells in the surface layers of the epithelium are predictive of disease grade. The risk associated with cellular abnormalities is stratified as follows:

A. High risk immediate treatment required. Positive for cellular proliferation markers; positive or negative for HPV infection. Indicative of a high grade lesion (Cancer, HSIL, CIN3 or CIN2)

B. Intermediate risk; early follow-up required. Positive for high-risk HPV type, but proliferation marker negative. Indicative of a low grade lesion (condyloma, LSIL or CIN1) caused by intermediate risk virus.

C. Lower risk; early follow-up recommended. Positive for intermediate risk HPV, but proliferation marker negative. Indicative of a low grade lesion (condyloma, LSIL or CIN1) caused by low risk virus.

D. Low risk; standard follow-up. Negative or low risk HPV; proliferation marker negative.

The invention accordingly further provides a method for assessing the risk associated with cellular abnormality in a patient sample, comprising obtaining a sample of the patient's cells; contacting said cells with two or more molecule(s), wherein at least one molecule is capable of binding a papilloma virus associated nucleic acid or antigen, and at least one molecule is capable of binding a cell proliferation marker or a plurality of cell proliferation markers and/or marker(s) of viral infection; and categorising the risk according to (a) the presence of cell proliferation markers, and (b) the detection of high or low risk HPV virus infection. The use of E4 as a marker is highly preferred over the use of nucleic acid, as it allows active virus infection to be examined whereas the use of DNA probes does not distinguish between active virus infection and latent infection. HPV DNA can be found in around 30% of women with normal cervical morphology and thus the presence of DNA does not provide an indication of disease status. E4 expression is inversely correlated with the severity of cervical disease. E4 is never expressed in tissue with normal histology.

### BRIEF DESCRIPTION OF THE DRAWINGS

FIG. 1A shows the amino acid sequence of HPV16 E4 protein and the binding sites of various antibody molecules or E4-specific antigen-binding fragments of antibodies;

FIG. 1B shows the sequence of the E4 protein from HPV16 (top row), HPV1 (bottom row) and a consensus sequence (middle row), and the binding sites of various antibodies or antigen-binding variants of antibodies;

FIGS. 2A-2D show four sensograms (arbitrary response units against time in seconds) obtained using surface plasmon resonance apparatus;

FIGS. 3-8 are micrographs showing variously stained samples, as explained in the text;

FIG. 9 is an amino acid sequence alignment of part of HPV E4 proteins;

FIG. 10 shows the distribution of E4 and MCM protein in a range of HPV16 induced HSIL. Panel (A): HSIL low (464); Low power image; brown=MCM; red=E4. Panel (B): HSIL (464); brown=MCM; red=16 E4. Panel (C): HSIL (11431); red=MCM; brown=E4. Panel (D): HSIL (11433): brown=MCM; red=16 E4. Panel (E): HSIL (15919); Brown=MCM; red=16 E4. In all images the nuclear signal indicates the presence of cellular MCM protein. Expression of MCM is induced by E2F and is absent from the surface layers of histologically normal epithelium as shown in other figures in this patent. MCM staining is abundant in HSIL due to the extensive expression of E7. The cytoplasmic signal present in the surface layers of some HSILs shown (464, 11433, 15919) indicates the presence of the viral E4 protein. Typically only a few cells at the epithelial surface are positive indicating that stimulation of the late stage of the virus life cycle is incomplete.

FIG. 11 shows the distribution of E4 protein and MCM in a range of HPV 16 induced LSIL. Panel (A): LSIL-low (2565) Low power image; red=MCM; brown=E4. Panel (B): LSIL (2565); red=MCM; brown=E4. Panel (C): LSIL ( 4165); red=MCM; brown=E4. Panel (D): LSIL (10451); red=MCM; brown=E4. Panel (E): LSIL (5388); red=MCM; brown=E4. The cytoplasmic staining in the upper epithelial layers is E4. The nuclear staining which is most abundant in the layers below those expressing E4, shows the distribution of MCM. The nuclear staining pattern for MCM extends above the basal layer but does not reach the surface. E4 expression is much more abundant in the surface layers of LSIL than MCM.

FIG. 12 shows double staining for junction region E4/MCM in the surface layers of normal cervical epithelium. Panel A: LSIL edge; red=MCM; brown=E4. Panel B: HSIL edge; red=E4; brown=MCM. Cytoplasmic staining in the surface layers is due to the presence of E4. Nuclear staining beneath this shows the distribution of MCM proteins. The two images show the junction between a LSIL and normal tissue or between a HSIL and normal tissue. The cytoplasmic E4 staining is absent in the normal tissue (to the right of each image) and the nuclear MCM signal is confined to cells in or close to the basal layer.

FIG. 13 shows combining staining for E4 in HPV1, HPV2 and HPV16 with MCM in immunofluorescence images. Panel A: HPV1 wart; red=MCM5; green=E4; blue=DAPI nuclear counterstain. Panel B: HPV2 wart; red=MCM5; green=E4; blue=DAPI nuclear counterstain. Panel C: HPV 16 LSIL; red=MCM5; green E4; blue=DAPI nuclear counterstain. E4 expression always follows MCM expression on low-grade lesions caused by other HPV types. Lesions caused by HPV1(wart), HPV2 (wart) and HPV16 (LSIL) are shown. In all cases, nuclear MCM staining is present in the lower epithelial layers whereas cytoplasmic E4 staining is found to begin in these cells as they migrate towards the epithelial surface. Cells positive for both E4 and MCM are present at the interface. As E4 expression coincides with the onset of genome amplification, this is likely to be a requirement for successful amplification of the viral genome.

FIG. 14 shows double-staining experiments which demonstrate that E2F-induced proteins can be substituted for MCM. Panel A: PCNA/E4 LSIL; Panel B: PCNA/E4 LSIL high power; Panel C: PCNA/E4 HSIL; Panel D: PCNA/E4 HSIL high power. All images red=PCNA, green=E4, blue=DAPI. Panel E: cyclin A/E4 LSIL; Panel F: cyclin A/E4 LSIL high power; Panel G: cyclin A/E4 HSIL; Panel H: cyclin A/E4 HSIL high power; Panel I: cyclin A/E4 HSIL 2; Panel J:cyclin A/E4 HSIL high power 2; All images, red=cyclin A, green=E4, blue=DAPI. The nuclear protein PCNA is activated by E2F and shows a similar expression pattern to MCM. Its expression (nuclear staining) is most extensive in the epithelial cell layers below those in which E4 expression (cytoplasmic staining) is abundant. Expression of PCNA in the surface layers of HSIL is more abundant than in the surface layers of LSIL. In HSIL, In HSIL, E4 expression is restricted to small pockets of cells which show some evidence of morphological differentiation.

Cyclin A is also activated by E2F. In cervical neoplasia caused by HPV16 it has a cytoplasmic and nuclear distribution and is found in sporadic cells in the basal epithelial layers and above. E4 (cytoplasmic staining) is detectable in the cytoplasm of cells in the layers above those that are expressing cyclin A. In HSIL, cyclin A expression extends into the surface epithelial layers, where as in LSIL, cyclin A rarely extends above the intermediate layer. As shown for MCM and PCNA, E4 expression in the surface layers is most abundant in regions where cyclin A expression is absent from the surface.

FIG. 15 is a further double-staining showing that other markers of cell proliferation can be substituted for E2F induced genes. Panel A: CyclinB/E4 LSIL. Panel B: cyclin B/E4 LSIL high power. Panel C: cyclinB/E4 HSIL. All images: red=cyclinB; green=E4, blue=DAPI. Both cyclin B and E4 show cytoplasmic staining patterns. In LSIL, cyclin B is found in the intermediate cell layers and below. E4 is expressed in sporadic cells in the intermediate layer and in layers above this. In HSIL, cytoplasmic cyclin B and E4 can both be found in cells near to the epithelial surface.

### DETAILED DESCRIPTION OF THE INVENTION

The Cervix

The cervical epithelium is essentially composed of two distinct cell types; the squamous epithelium and the columnar epithelium, each of which is located in an anatomically distinct region of the tissue. The squamous epithelium is located at the exterior aspect (the ectocervix) of the cervical opening (os), while the columnar epithelium extends into the endocervical canal (the endocervix). These two distinct epithelial cell types come into contact in the vicinity of the cervical os, the squamo-columnar junction. The squamo-columnar junction is of clinical importance as it is the region where the majority of malignancies arise. For diagnostic validity, a cervical smear sample should include cells from this region. In order to ensure that this has been achieved, a smear should contain columnar as well as squamous epithelial cells.

Most cervical tumours arise at the squamo-columnar junction from the squamous epithelium, which is multilayered dynamic stem cell system under constant renewal. The stem cell compartment itself is located adjacent to the basement membrane within the basal cell layer. Stem cell division gives rise to parabasal, intermediate, and superficial cell derivatives. These are conventionally defined in terms of both their characteristic morphology and location within the squamous epithelium. The transition from basal cells located in the deepest layer of the squamous epithelium, to superficial cells at its surface is associated with progressive differentiation and a loss of proliferation until superficial squamous epithelial cells at the cervical surface are terminally differentiated.

In dysplasia, there is increased cellular proliferation with a reduction in differentiation of cells as they progress through the squamous epithelium. Typically, for convenience in the first instance, cervical screening involves assessment of smears taken from the surface of the epithelium, looking for abnormalities at the surface representative of reduced differentiation as a result of dysplasia.

At the late foetal stage, during adolescence and in pregnancy columnar epithelium is replaced at the junction by squamous epithelium by a process of metaplasia. Metaplastic squamous cells which replace columnar cells are particularly vulnerable to carcinogens. Normal metaplasia should not be confused with abnormal dysplasia within the squamous epithelium. The majority of cases of cervical malignancies represent squamous cell carcinoma (SCC) and are strongly associated with infection with ‘high-risk’ types of human papillomavirus, such as 16, 18 and 31 (Park et al. Cancer, 1995, 76 (10 Suppl.) : p. 1902-13). Cervical carcinoma is amenable to prevention by population screening, as it evolves through well-defined non-invasive ‘intraepithelial’ stages (Wright, et al. Precancerous lesions of the cervix, in Blaustein's pathology of the female genital tract. R. J. Kurman, Editor. 1994, Springer-Verlag: New York. p. 229-78. Squamous intraepithelial abnormalities may be classified using 3 tier (CIN) or 2 tier (Bethesda) systems. Different histological abnormalities broadly correlate with the type of infecting HPV and with the DNA ploidy, clonality and natural history of the lesion. As classified by the Bethesda system, low grade squamous intra-epithelial lesions (LSIL), corresponding to CIN1 and cervical HPV infection (HPVI) generally represent productive HPV infections, with a relatively low risk of progression to invasive disease (Wright and Kurman. A critical review of the morphological classification systems of preinvasive lesions of the cervix: the scientific basis for shifting the paradigm, in Papillomavirus reviews: current research on papilloniaviruses, C Lacey, Editor. 1996, Leeds University Press: Leeds). High grade squamous intra-epithelial lesions (HSIL), corresponding to CIN2 and CIN3, show a higher risk of progression than CIN1 (LSIL) though both are viewed as representing potential precursor of malignancy. Although it is possible to estimate the approximate risk of malignancy for each category of intra-epithelial lesion, it is currently not possible to determine the approximate likelihood of progression for an individual case.

The major reasons why cytological screening sometimes fails to detect cervical cancer are the large intervals between tests and also the high number of false negative results (10-30%) (Pap Cytology screening: Most of the benefits reaped? WHO and EUROGIN release a report on cervical cancer control. Press Release WHO/25, March 1997).

The high number of false negative results reflects the fact that interpretation of Pap smears is one of the most difficult of morphological exercises. The results of a Pap smear are harder to interpret than those of fine needle aspiration, body fluid cytological testing or biopsies because of the complexity and variability of the mixed cell population placed on the smear and the wide range of inflammatory and reparative processes that occur in the cervix. There are also cyclical changes in the cellular population, pregnancy induced alterations and alterations that occur in the postmenopausal period. Because gynaecological cytology is so difficult, the training periods for cytotechnologists are long; they require an educated student and high degree of discipline and pattern recognition skills. Even after completing an adequate training programme, cytotechnologists require several years of practical experience before they can make consistently accurate judgements as to whether a Pap smear result is normal or abnormal. Similarly, although pathologists may be trained to interpret histological sections, they require specialised additional training in cytopathology to possess adequate skills to organise and supervise the cytology laboratory and to make appropriate diagnoses concerning abnormal smears.

The two major problems associated with current screening programmes are an apparently unavoidable false negative rate (10-30%) and the relatively high cost of screening. Therefore, alternative approaches are now being considered to cervical screening. The two most commonly discussed proposals are to use HPV DNA testing and typing as a primary screening modality or as a supplement to Pap smears and to use instruments that can automatically screen conventionally taken Pap smears, thus reducing the need for the relatively highly paid cytotechnologists and cytopathologists (Richart, Cancer Supplement, 1995, 76(10): 1919-1927; Birdsong, Human Pathology, 1996, 27(5): 468-481).

A number of companies are currently developing and marketing automated screening instruments. In general such instruments use a high resolution video scanner to capture images, which are then digitised and analysed with a series of algorithms, and the data are then passed through an interference network through which the machine has been trained to distinguish between normal and abnormal cellular components. It is hoped that with further software and hardware development, automated screening can be considered for primary screening, though at the moment no devices have been approved for the pre-screening or independent screening of Pap smears by the US FDA. That companies are prepared to invest so heavily in such an expensive and complex approach in attempting to overcome problems with conventional PAP smear testing illustrates the severity of the problems and the long-felt need for a solution.

Cell Proliferation Markers

Measuring parameters of cell proliferation can provide objective information about tumours, for example using cell proliferation markers such as PCNA, Ki67 etc. The most widely studied markers of proliferation are Ki67 and PCNA (proliferating cell nuclear antigen) (Yu and Filipe, Histochemcial Journal, 1993, 25: 843-853). PCNA is involved in the elongation of DNA replication and in the mechanism of DNA repair. Therefore it is present during actual DNA synthesis by replication or repair.

There are also useful markers involved in the earlier initiation stage of DNA replication. These include Cdc6 and proteins of the MCM2-7 family (MCM2, MCM3, MCM4, MCM5, MCM6 and MCM7). Williams et al (1997) (Proc. Natl. Acad. Sci. USA, 1997, 94: 142-147) reported that human HeLa Cells in culture express Cdc6 throughout proliferating cell cycles, but that W138 human diploid fibroblasts stop expressing Cdc6 when made quiescent by serum starvation. It is shown herein that these observations extend to other cell lines and other species. MCMs are present in G1 phase nuclei (before DNA synthesis) and are progressively displaced from chromatin into the soluble nucleoplasm during DNA synthesis. It is shown that they are absent from chromatin during quiescence. It is also known that MCM5 is absent from differentiated cells of the uterine cervix and breast. Cdc6 antibodies or MCM (e.g. MCM5) antibodies are known to detect LSIL (HPVI/CIN 1) lesions of the cervix. Furthermore, essentially all cells of LSIL (HPVICIN 1) or HSIL (CIN 2/3) lesions are stained. This indicates that specific binding molecules directed to proteins of the preinitiation complex of DNA replication, particularly Cdc6 or MCM proteins (such as MCM5, MCM2, MCM3, MCM4, MCM6 and MCM7) are useful for detection of atypical or neoplastic cells.

Useful cellular proliferation markers may include human homologues of yeast components, such as Cdc7 protein kinase (Chapman and Johnston, Exp. Cell Res., 1989, 180 419-428 (yeast), Sao et al. 1997, EMBO J., 16, 4340-4351 (human—down-regulated in quiescence)), Dbf4, the regulatory subunit of Cdc7 protein kinase (Jackson et al., 1993, Mol. Cell Biol. 13 2899-2908 (yeast), Masai et al., Cold Spring Harbor Meeting on Eukaryotic DNA Replication, 3-7 September 1997 (human)), Cdc14 protein phosphatase (Hogan and Koshland PNAS USA, 1992, 89, 3098-3102 (yeast)), Cdc45, which associates with and has a similar phenotype to MCMs (Zou et al., Mol. Cell Biol., 1997, 17, 553-563 (yeast), Takisawa et al., Cold Spring Harbor Meeting On Eukaryotic DNA Replication, 3-7 Sep. 1997 (Xenopus)), MCM10, which associates with and has a similar phenotype to MCMs (Merchant et al., 1997, Mol. Cell Biol. 17 3261-3271) as well as replication factors such as ORC2. Target polypeptides of the present invention may variously be said to be any of components of the DNA pre-replicative complex, components of replication competent chromatin, involved in restricting DNA replication to once per cell cycle, components of the replication licence, involved in licensing chromatin for a single round of DNA replication, and assembled at replication origins before initiation of DNA replication.

Moreover, both cyclin A and the viral activity marker cyclin B are useful markers in the invention, as is p 16.

The sequence of human cyclin B mRNA is disclosed in Jackman et al., EMBO J. 14 (8), 1646-1654 (1995); see GenBank Acc. No. NM_{—}004701.2 GI:10938017.

The human cyclin A mRNA sequence is available on GenBank accession no. XM_{—}003325.1 GI:11446079.

The sequence of human p16 is available on GenBank accession no. XM_{—}005656.1 GI: 11429662.

Human Cdc6 amino acid sequence is disclosed in Williams et al., 1997, PNAS USA 94: 142-147, GenBank Acc. No. U77949.

Human MCM2 sequence is disclosed in Todorov et al., 1994, J. Cell Sci., 107, 253-265, GenBank Acc. No. X67334.

Human MCM3 sequence is disclosed in Thommes et al., 1992, Nucl. Acid Res., 20, 1069-1074, GenBank Acc. No. P25205.

Human MCM4 sequence is disclosed in Ishimi et al., 1996, J. Biol. Chem., 271, 24115-24122, GenBank Acc. No. X74794.

Human MCM5 sequence is disclosed in Hu et al., 1993, Nucleic Acids Res., 21, 5289-5293, GenBank Acc. No. X74795.

Human MCM6 sequence is disclosed in Holthoff et al., 1996, Genomics, 37, 131-134, GenBank Acc. No. X67334.

Human MCM7 sequence is disclosed in Hu et al., 1993, Nucleic Acids Res., 21, 5289-5293.

Human Cdc6 has been cloned by Williams et al. whose paper (PNAS USA 94: 142-147, 1997) provides the full amino acid sequence. Anti-Cdc6 binding molecules are very effective in marking abnormality in various tissues, especially cervical samples, preferably smears. This compares with no binding to normal cervical tissue in a smear sample.

The amino acid sequence for human MCM5 is disclosed in Hu et al., 1993, Nucleic Acids Res., 21, 5289-5293, GenBank Acc. No X74795. Binding molecules directed against it, like Cdc6, are very effective in marking abnormality in various tissues, especially cervical samples, preferably smears.

Binding molecules directed against MCM2, against MCM3, against MCM4, against MCM6 or against MCM7 are also effective in marking abnormality in tissue samples such as cervical smears.

Thus, binding of (e.g.) an anti-Cdc6 or anti-MCM specific binding member to a sample provides for categorising the tissue from which the sample is derived as abnormal, potentially or actually pre-cancerous, dysplastic or neoplastic. In accordance with present practice upon obtaining a positive result using the Pap test, a positively-testing individual may be investigated further, for instance by means of biopsy testing and/or repeat screening. It is quite common for pre-cancerous potential not to result in an actually cancerous state. Six-monthly screening is typically used to follow progression or regression of dysplasia to allow for appropriate and timely therapeutic intervention if required.

The present invention may be used to pre-screen samples before further analysis. The present invention may be used for screening or analysis of samples previously tested using an available technique, such as a Pap smear test or ThinPrep 2000 test. Thus, a cervical smear for example may be tested using both the conventional Pap smear test and a test in accordance with the present invention.

In a further aspect the present invention provides a method for determining, assessing or diagnosing the presence or absence of abnormal cellular proliferation, cellular growth abnormality, dysplasia, neoplasia, or a potentially or actually pre-cancerous or cancerous state in a tissue or sample thereof.

Kits

Binding molecules may be provided in a kit, which may include instructions for use in accordance with the present invention. Such kits are provided as a further aspect of the present invention. One or more other reagents may be included, such as labelling molecules, and so (see below). Reagents may be provided within containers which protect them from the external environment, such as a sealed vial. A kit may include one or more articles for providing the test sample itself depending on the tissue of interest, e.g. a spatula for taking a cervical smear, (such components generally being sterile). A kit may include any, any combination of, or all of; a blocking agent to decrease non-specific staining, a storage buffer for preserving binding molecule activity during storage, staining buffer and/or washing buffer to be used during antibody staining, a positive control, a negative control. The design and use of controls is standard and well within the routine capabilities of those of ordinary skill in the art.

Samples

Samples may be removed from the body using any convenient means and technique. For cervical screening, standard smear samples may be employed. Alternatively, the ThinPrep 2000 technology (Cytec Corp, Boxborough, Mass., USA) may be used. This has been cleared by the US FDA as a replacement for the conventional method of Pap smear preparation. A sample is collected into a liquid medium instead of smearing the cells onto a glass slide. An automated processor (the ThinPrep 2000 machine) is later used to collect the cells from the liquid and deposit them in a thin layer on a glass slide for analysis. A spatula or swab may be used to remove endothelium cells from the cervix.

The binding of molecules such as an antibody to samples may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding binding molecule (e.g. antibody) and reporter molecule.

One favoured mode is by covalent linkage of each binding member with an individual fluorochrome, phosphor or laser dye with spectrally isolated absorption or emission characteristics. Suitable Fluorochrome include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed. Further examples are horseradish peroxidase and chemiluminescence.

Those skilled in the art are able to choose a suitable mode of determining binding according to their preference and general knowledge. For example, horseradish peroxidase or alkaline phosphatase, may be used.

Papilloma Viruses

Studies on naturally-occurring warts have revealed the virus to encode three late proteins—L1 and L2, which are virion coat proteins (Doorbar et al, 1987), and E1\hat{ }E4, a non-stnictural late protein of unknown function (Doorbar et al, 1986). In HPV1-induced warts the E1\hat{ }E4 protein is first expressed in cells of the lower spinous layer, and assembles into distinctive cytoplasmic and nuclear inclusions. During terminal differentiation it is post-transcriptionally modified by phosphorylation (Grand et al, 1989) and by removal of sequences from the N-terminus (Doorbar et al, 1988; Roberts et al, 1994). The E1\hat{ }E4 proteins of high risk viruses have been characterised, for example in WO98/25145. Of these viral proteins, E1\hat{ }E4 is found to be much more abundant than either L1 or L2 where it has been estimated (Doorbar et al 1987). Moreover, E1\hat{ }E4 precedes both L1 and L2 in its expression and its expression is preserved into high grade lesions even when L1 is lost.

The sample of patient's cells may comprise skin cells (e.g. in the case of warts, veruccas and the like, caused by cutaneous HPV infections). Cutaneous lesions, such as those induced by HPV types 5, 8, 14, 17, 20, are difficult to manage clinically, and are often associated with malignancies in immunosuppressed patients (Benton et al, 1992 Papillomavirus Reports 3, 23-26). Alternatively, the sample may comprise mucosal cells, especially cervical cells, in the case of HPV infections of the urinogenital tract. Methods of obtaining and preparing such samples for use in the method of the invention are known to those skilled in the art or will be apparent from the present disclosure.

The term “pre-cancerous cervical lesions” is intended to refer to those abnormalities which clinically may be described as “pre-malignant” conditions and which may, without treatment, proceed to full malignancies. As set forth above, such lesions are screened for routinely by, for example, cervical smear testing. The present invention allows for cells obtained from patients by methods such as cervical smears to be tested more accurately and more quickly for HPV infection, simultaneously with testing for the later stages of malignancy.

Screening for Nucleic Acids

Methods for screening cells for nucleic acids derived from HPV are well known in the art. For example, a test known as “Hybrid Capture” is available from Digene (www.digene.com) for the detection of a number of organisms, including HPV. Current “Hybrid Capture 2” technology is described in literature available from Digene. Hybrid capture technology relies on RNA probes which hybridise to target DNA present in infected cells. The DNA/RNA hybrids are detected using alkaline phosphatase conjugated antibodies and a chemiluminescent substrate.

Nucleic acid hybridisation using DNA or RNA probes is generally applicable to the detection of HPV. Alternative systems may rely, for example, on PCR of HPV nucleic acids using appropriate primers.

Antibodies

Preferably, a molecule according to the invention protein comprises an antibody molecule or an antigen-binding variant thereof, such as a Fab, Fv, scFv, “diabody” and the like. The molecule may comprise monoclonal or polyclonal antibodies, or antigen-binding portions of antibodies selected from libraries by screening (e.g. using phage display technology). Alternatively the molecule may be some other polypeptide, peptide, a synthetic compound or an RNA or DNA aptamer, generated by a procedure such as SELEX. In some preferred embodiments the molecule comprises a label moiety, such as a fluorophore, chromophore, enzyme or radio-label, so as to facilitate monitoring of binding of the molecule to E4 protein. Such labels are well-known to those skilled in the art and include, for example, fluorescein isothiocyanate (FITC), β-galactosidase, horseradish peroxidase, streptavidin, biotin, ³⁵S or ¹²⁵I. Other examples will be apparent to those skilled in the art. The label may in some instances be conjugated to the antibody or antigen-binding variant, or may be present (where the label is a peptide or polypeptide) as a fusion protein.

Preferably at least some of the molecules used in the method of the invention bind selectively to the E4 protein of a certain HPV type or types, but not to the E4 protein of other HPV types. Accordingly, in one embodiment the invention can be used to determine the type or types of HPV infecting a patient. This is very significant, as progression to malignant disease (and hence clinical prognosis) is heavily dependent on HPV type. Accordingly, the invention provides a method of determining the type(s) of HPV infection in a patient, the method comprising the steps of: obtaining a sample of the patient's cells from the site of HPV infection; contacting the cells with a molecule that binds specifically to a subject of HPV E4 proteins; and monitoring said binding. Preferably, said molecules bind type-specifically to the E4 polypeptide, allowing the identification of high-risk HPV types.

In a further aspect the invention teaches the use of an antibody molecule, or an antigen-binding variant thereof, which binds specifically to HPV E4 protein in the region of amino acid residues RPIPKPSPWAPKKHRRLSSDQDSQTP of HPV16 E4 protein, or the corresponding hydrophilic, acid/base-rich region of other HPV E4 proteins.

In a method according to the invention, the subset of E4 proteins to which at least one molecule binds may consist of a single HPV type E4 protein, or may consist of a plurality of E4 proteins of different types. If the E4 proteins to which the molecule binds do not encompass the E4 proteins of all known HPV types, then binding or non-binding (as appropriate) of the molecule to the E4 protein present in the cell sample may allow an investigator to make certain deductions about the identity of the HPV type(s) infecting the patient.

In practice it may be advantageous to employ a plurality of different molecules, which bind to different HPV proteins. This may be helpful in identifying the type(s) of HPV infecting a patient, although it is not essential as a prognostic indicator. For example, the ability to limit the infecting HPV type(s) to a particular subset (or exclude such a subset) may be advantageous. By way of explanation, it is known that mucosal HPV types 6, 11, 42, 43 and 44 are associated with external genital papillomas (condylomata accuminata) which have a low risk of progression to cancer, but are difficult to eradicate and are disruptive to the lives of the patients. The higher risk mucosal types (31, 33, 35, 51, 52, 58, 61 and 16, 18, 45, 56) cause asymptomatic flat warts (flat concyloma) which can progress to high grade cervical intraepithelial neoplasia (CIN) and cancer. The highest risk of progression to malignancy is associated with lesions caused by HPV types 16, 18, 45 and 56.

Different probes specific for different subtypes of HPV may be labelled with different labels. For example, high-risk HPV-specific probes may all be labelled with a single label; low risk HPV-specific probes may be labelled with a single but different label. The test operator would then easily be able to determine the risk associated with the sample according to the label. Fluorescent labels would be particularly advantageously employed in such circumstances.

Sorting of cells into risk categories according to label may be done in a variety of ways, and may be automated, using detectors sensitive to differences in label emissions, such as fluorescence frequency and/or intensity. In a particularly advantageous embodiment, cells may be sorted by FACS; a prevalent distribution of cells into a high-risk group indicates the presence of infection by high-risk HPV.

Molecules which bind to particular HPV types, but not to other HPV types, may be generated for example by randomization and selection techniques. These include phage display, and other techniques suitable for displaying antibodies or other polypeptides; and procedures for generating nucleic acid binding molecules, for example RNA aptamers, such as SELEX. These procedures are well known to those of ordinary skill in the art and described below for the purposes of exemplification. The invention accordingly provides HPV-binding molecules targeted to the HPV E4 protein, which are useful in methods as described herein.

According to the present invention, HPV binding molecules are preferably targeted to extracellular portions of the HPV E4 polypeptide. Such portions tend to be hydrophilic in character. Preferably, therefore, at least some of the molecules according to the invention specifically bind to hydrophilic portions of the HPV E4 protein.

The present invention moreover describes a particular region of the E4 protein to which molecules (particularly antibody molecules or variants thereof) may bind with considerable specificity. Although homologous regions exist in all HPV E4 proteins, the region varies in amino acid sequence between HPVs of different types. The region corresponds to a peak of hydrophilicity in the E4 protein and is probably surface-exposed. The region is highly charged (acid/base-rich). In HPV type 16, the amino acid sequence of the region is (from N-terminal to C-terminal) RPIPKPSPWAPKKHRRLSSDQDSQTP. Clearly the amino acid sequence of the E4 proteins of other HPV types will not necessarily be identical to that in type 16, but the corresponding region can readily be identified in other E4 proteins by those skilled in the art by use of conventional alignment and sequence comparison computer programs (about 65 of the 70 or so known HPV genomes have been cloned and sequenced).

Thus, the invention also involves an antibody molecule, or an antigen-binding variant thereof, which binds specifically to HPV E4 protein in the region of amino acid residues RPIPKPSPWAPKKHRRLSSDQDSQTP of HPV16 E4 protein, or the corresponding hydrophilic, acid/base-rich region of other HPV E4 proteins, preferably other than the antibody TVG 402 identified by Doorbar et al, (1992 Virology 187, 353-359).

Moreover, the invention involves the use of an antibody molecule, or an antigen-binding variant thereof, which binds specifically to HPV E4 protein in the region of amino acid residues RPIPKPSPWAPKKHRRLSSDQDSQTP of HPV16 E4 protein, or the corresponding hydrophilic, acid/base-rich region of other HPV E4 proteins for the detection of HPV infections as described herein.

The corresponding hydrophilic acid/base-rich regions of large numbers of different HPV types are shown in FIG. 9. FIG. 9 shows a consensus-type amino acid sequence (“most likely”) on the top row, with the sequence of HPV E4 proteins below. Dots indicate gaps introduced to facilitate the alignment, dashes denote amino acid residue matches with the consensus sequence. Numbering on the right hand side of the figure indicates the number of amino acid residues from the actual or predicted E1\hat{ }E4 splice site. It will be appreciated by those skilled in the art from the alignment that whilst the hydrophilicity of the region is conserved amongst different HPV types, the actual amino acid sequence varies quite considerably, such that reagents binding to this region may be expected to be highly HPV type-specific.

Preferably an antibody suitable for use in the invention has a binding site, as identified by the SPOTS epitope mapping system, within the region RRIPKPSPWAPKKHR (or the corresponding amino acid sequence from other HPV types). A particularly preferred molecule is the Fab fragment TVG405, described further below, which binds to the epitope PKPSPWAPKKH(R) with extremely high affinity and is of particular usefulness in the methods of the invention defined above.

The arginine residue indicated in brackets at the C-terminal of the TVG405 epitope is not essential for high affinity binding.

The Fab fragment TVG405 was isolated by the present inventor using phage display technology, as described below. Those skilled in the art will understand that different antibodies or Fab fragments may readily be obtained by using similar phage display techniques (and screening with relevant polypeptides or portions thereof), or by using more conventional immunisation techniques (e.g. immunising mice, rabbits, rats or the like with HPV or cell proliferation proteins, or peptides corresponding to portions of such polypeptides) to obtain polyclonal antisera or monoclonal antibodies (using well known hybridoma techniques of Milstein et al). Complete antibody molecules can readily be prepared from Fab—encoding sequences (e.g. isolated by phage display techniques) using standard DNA manipulation techniques described by Sambrook et al, (Molecular Cloning: A Laboratory Manual, 2nd Edition, Cold Spring Harbor Laboratory Press, NY, USA) to join appropriate DNA sequences.

Similarly, standard DNA manipulative techniques can be used to modify DNA sequences encoding antibodies or antigen-binding variants thereof. In particular site-directed mutagenesis or PCR can be used to modify the coding sequences, so as to produce modified anti-HPV or anti-cell proliferation marker antibodies with different binding specificities or affinities. Alternatively fusion proteins, comprising the binding site of an Fab, Fv or antibody and the like, may be prepared.

Molecules capable of binding HPV polynucleotides or polypeptides or cell proliferation markers may be used as anti-viral or anti-cancer agents, or parts of such agents. For example, antibody molecules or E4-binding peptide as described above may be employed for this purpose. Preferably, however, the E4 protein and/or molecules capable of binding thereto may be used to design E4-binding molecules, preferably small molecules, by rational drug design. The invention moreover concerns the use of molecules capable of binding to E4 to target antiviral agents capable of destroying papilloma viruses and/or cells infected by papilloma viruses. Such molecules may be antibodies or peptides as described above and exemplified herein, optionally conjugated to anticancer or antiviral agents.

Such a process preferably involves the crystallisation of the relevant polypeptide or a molecule capable of binding thereto. More preferably, such a process involves the co-crystallisation of the polypeptide and a binding agent. Such a procedure gives information concerning the interaction between the polypeptide and the binding molecule, which can be used to design small molecules capable of mimicking the binding interaction.

Crystallisation involves the preparation of a crystallisation buffer, for example by mixing a solution of the peptide or peptide complex with a “reservoir buffer”, preferably in a 1:1 ratio, with a lower concentration of the precipitating agent necessary for crystal formation. For crystal formation, the concentration of the precipitating agent is increased, for example by addition of precipitating agent, for example by titration, or by allowing the concentration of precipitating agent to balance by diffusion between the crystallisation buffer and a reservoir buffer. Under suitable conditions such diffusion of precipitating agent occurs along the gradient of precipitating agent, for example from the reservoir buffer having a higher concentration of precipitating agent into the crystallisation buffer having a lower concentration of precipitating agent. Diffusion may be achieved for example by vapour diffusion techniques allowing diffision in the common gas phase. Known techniques are, for example, vapour diffusion methods, such as the “hanging drop” or the “sitting drop” method. In the vapour diffusion method a drop of crystallisation buffer containing the protein is hanging above or sitting beside a much larger pool of reservoir buffer. Alternatively, the balancing of the precipitating agent can be achieved through a semipermeable membrane that separates the crystallisation buffer from the reservoir buffer and prevents dilution of the protein into the reservoir buffer.

In the crystallisation buffer the peptide or peptide/binding partner complex preferably has a concentration of up to 30 mg/ml, preferably from about 2 mg/ml to about 4 mg/ml.

Formation of crystals can be achieved under various conditions which are essentially determined by the following parameters: pH, presence of salts and additives, precipitating agent, protein concentration and temperature. The pH may range from about 4.0 to 9.0. The concentration and type of buffer is rather unimportant, and therefore variable, e.g. in dependence with the desired pH. Suitable buffer systems include phosphate, acetate, citrate, Tris, MES and HEPES buffers. Useful salts and additives include e.g. chlorides, sulphates and further salts specified in Example 1. The buffer contains a precipitating agent selected from the group consisting of a water miscible organic solvent, preferably polyethylene glycol having a molecular weight of between 100 and 20000, preferentially between 4000 and 10000, or a suitable salt, such as a sulphates, particularly ammonium sulphate, a chloride, a citrate or a tartrate.

A crystal of a peptide of interest, or a peptide/binding partner complex according to the invention may be chemically modified, e.g. by heavy atom derivatization. Briefly, such derivatization is achievable by soaking a crystal in a solution containing heavy metal atom salts, or a organometallic compounds, e.g. lead chloride, gold thiomalate, thimerosal or uranyl acetate, which is capable of diffusing through the crystal and binding to the surface of the protein. The location(s) of the bound heavy metal atom(s) can be determined by X-ray diffraction analysis of the soaked crystal, which information may be used e.g. to construct a three-dimensional model of the peptide.

A three-dimensional model is obtainable, for example, from a heavy atom derivative of a crystal and/or from all or part of the structural data provided by the crystallisation. Preferably building of such model involves homology modelling and/or molecular replacement.

The preliminary homology model can be created by a combination of sequence alignment with any of the proteins of which the sequence is known, secondary structure prediction and screening of structural libraries. For example, the sequences of HSV 16 and 34 E4 can be aligned as set forth herein.

Computational software may also be used to predict the secondary structure of peptides or peptide complexes. The peptide sequence may be incorporated into the structure. Structural incoherences, e.g. structural fragments around insertions/deletions can be modelled by screening a structural library for peptides of the desired length and with a suitable conformation. For prediction of the side chain conformation, a side chain rotamer library may be employed.

The final homology model is used to solve the crystal structure of peptides by molecular replacement using suitable computer software. The homology model is positioned according to the results of molecular replacement, and subjected to further refinement comprising molecular dynamics calculations and modelling of the inhibitor used for crystallisation into the electron density.

Similar approaches may be used to crystallise and determine the structure of HPV-binding or cell proliferation-binding polypeptides, including antibodies and antibody fragments, for example those used in the present invention.

It has surprisingly been found that E4 expression correlates strongly with vegetative DNA replication in HPV-infected cells, making detection of E4 expression a particularly appropriate indicator of HPV infection, and thus particularly useful in screening for precancerous cervical lesions.

Other available methods of cervical screening by HPV detection are based on DNA hybridisation. They involve cell lysis or permeabilisation and are performed in an ELISA-type 96 well format. The hybridisation is ultimately visualised as a colour change in one of the wells.

Although the antibodies of the present invention could be used in a similar way (i.e. following cell lysis), they are amenable to a quicker procedure which would be more readily carried out routinely by histopathology laboratories. Samples comprising cervical cells may be taken as usual. These are be spread for example on a microscope slide or other support using techniques known in the art, for example as exemplified herein, and stained with, for example, an anti-E4 Fab. Detection may be performed with a secondary antibody-enzyme conjugate (horseradish peroxidase, alkaline phosphatase), or the Fab could be directly conjugated, for example to a fluorophore, such as FITC. This approach may be adapted for use with systems that are currently available for increasing the sensitivity of antibody detection. At present, cervical smears are examined routinely by microscopy. The proposed approach would require no new equipment and could easily fit around existing methods.

Staining of Cervical Smears

One protocol for antibody staining of cervical smears is as follows.

Fix fresh smear for 5 minutes in 50:50 acetone:methanol. (Note than an alternative starting point where a smear has been previously fixed with “Cytofix”—an alcoholic and wax treatment that is in standard use in the UK to treat smear samples when taken to allow safe transport to a screening centre—is to remove Cytofix by soaking in methylated spirits for 10 minutes. Should a smear have been covered with any other protective layer, any appropriate treatment may be employed to expose a sample to antibody staining).

Wash in Tris-buffered saline (TBS) for 5 minutes.

Wash to permeabilise in 4 mM sodium deoxycholate in TBS for 15 minutes.

Wash in TBS plus 0.3% Triton X100 for 5 minutes.

Repeat step 4.

Wash in TBS plus 0.025% Triton X100 for 5 minutes.

Drain excess liquid without allowing tissue to dry out.

Transfer slides into a humidified chamber and place on each slide 200 microlitres of 10% goat serum reagent in TBS for a minimum of 2 hours (or overnight).

Drain excess liquid without allowing tissue to dry out.

Place 200 microlitres of primary antibody diluted in TBS containing 0.1% Triton and 1% BSA onto each slide and leave overnight at 4° C. on an orbital shaker.

Transfer slides into racks and wash in TBS plus 0.3% Triton X100 for 5 minutes.

Wash in TBS plus 0.025% Triton X100 for 5-minutes.

Repeat step 12.

Drain excess liquid without allowing tissue to dry out.

Transfer slides into a humidified chamber and place on each slide 200 microlitres of biotinylated goat anti-rabbit secondary antibody (Dako) at 1:500 in TBS containing 1% BSA for 1 hour.

While slides are in secondary antibody, male up SABC solution.

Transfer slides into racks and wash in TBS for 5 minutes.

Place slides in endogenous peroxidase blocking agent with 0.6% hydrogen peroxide for 10 minutes.

Wash in TBS for 5 minutes.

Repeat step 19 twice.

Transfer slides into a humidified chamber and place on each slide 200 microlitres of SABC solution for 30 minutes.

Transfer slides into racks and wash in TBS in 5 minutes.

Repeat step 22.

Develop in DAB solution for 10 minutes.

Wash in running tap water for 5 minutes.

Place slides in Harris' haematoxylin solution for 6 seconds.

Wash in running tap water for 1 minute.

Differentiate in 0.5% hydrochloric acid for 1-2 seconds.

Wash in running tap water for 5 minutes.

Rinse in 50% methanol for 2 minutes.

Rinse in 70% methanol for 2 minutes.

Rinse in 90% methanol for 2 minutes.

Rinse in 100% methanol for 2 minutes.

Place in Orange G working solution for 2 minutes.

Rinse in 100% methanol for 7 seconds and agitate gently.

Repeat step 35.

Place in EA50 solution for 2 minutes.

Rinse in 100% methanol for 7 seconds and agitate gently.

Repeat step 38.

Place slides in xylene to clear for 5 minutes.

Repeat step 40 twice.

Apply coverslips using DEPEX mountant.

Smears for immunofluorescence may be prepared in a similar fashion. After the secondary antibody, they are incubated in streptavidin FITC-conjugated antibody for 1 hour and counterstained for DNA with propdium iodide/RNAse A (both Sigma at 50 ng/ml), then washed and mounted in glycerol/PBS/phenylenediamine.

Preferred binding molecules for use in aspects of the present invention include antibodies, natural ligands for the target and T-cell Receptor binding domains.

The present invention provides a new approach to screening for cervical abnormalities.

The prior art in this field comprises diagnostic tests for the presence of papilloma viruses which were previously developed by us (eg. WO98/25145). These rely on detection of viral constituents, principally using antibodies, and use this information about the state of viral infection as an indication of the likelihood of developing higher grade lesions or malignancies preceding cervical cancer.

A different and unconnected prior art document teaches an approach to screening for malignancies by examining tissue samples such as blood, biopsies, body fluids etc, including cervical samples, for abnormally proliferating cells (WO99/21014). This relies on the detection of human cell cycle expressed polypeptides, which are usually intracellular, and provides an indication of the presence of human cell lineages which are proliferating ectopically.

Clearly, although these two approaches share some biochemical elements such as the use of immunological reagents, they are very different in principle and in practice. The former is based on collecting of information about viral life cycles, and characterisation of particular sub-types of viral infection, and relating this to the probability of more serious lesions developing. The latter test, on the other hand, is strictly concerned with the monitoring of the cell division state of human cells from various sources, and aims to relate this information to the presence or absence of malignancies in the sample.

The present invention combines these two different tests to provide a much simpler approach which yields more information more quickly than would conventional pap-smear testing programmes, or than either test applied individually.

The present invention is founded in double and triple stains in which antibodies to E4 have been combined with antibodies to genes such as PCNA, MCM and cyclinA which are present in cervical lesions because they are induced by E7. Thus these proteins can be considered to be surrogate markers of E7 activity. The prior art does not connect the expression of MCM in cervical lesions with the fact that such genes are, in effect, markers of E7 activity. The analysis of E4 and MCM in productive infections caused by HPV 1, HPV2 and HPV11 reveals a common pattern of expression in productive infection which changes in abortive infections caused by HPV16. This could not have been predicted from previous studies which did not make use of the double staining approach.

It is an advantage of the present invention that the same sample can be used to provide much more information than a single individual test might.

By screening for viral infection and for higher grade malignancies in the same test, only one sample needs to be obtained for a much broader picture of the patient's condition to be obtained.

By screening for ‘early’ events which may lead to cervical lesions (i.e. HPV infections) whilst simultaneously examining the sample for later stages of malignancy, a better prognosis may be obtained.

Furthermore, it is a known problem that conventional smear testing can produce a high proportion of false negatives, whereas by combining two entirely different tests into the single procedure of the present invention, the chances of such false negatives occurring is much reduced.

A further advantage of the present invention is that fewer samples are needed to gain the same amount of information. For example, if a patient was discovered to have a HPV infection, the doctor may need to recall that patient for further examination, whereas by employing the methods of the present invention, more information is gained in the first instance, thereby reducing the burden on staff performing the tests, and those interpreting them.

Moreover, patients do not generally find the smear test procedure comfortable. Many find it embarrassing and unpleasant, and therefore by using the methods of the present invention, which may alleviate the need for some repeat or further testing, patient suffering may be directly reduced by requiring fewer samples to be collected.

By simultaneously testing the same sample for more than one condition, there is introduced into the procedure a robust means for cross-comparison between the tests. For example, relative staining intensities might be meaningfully compared where they might not have been if the tests had been performed independently on different samples. This is another example of the synergistic advantages offered by the methods of the present invention.

One of the problems in cervical screening, particularly in developed countries, is the often long periods which some women experience between tests. This could mean that by testing for only early stages of the disease, these may have already passed by the time a sample is examined. Equally, if only the late stages of the disease are monitored, then a patient may be correctly given a negative test, but develop the disease during the intervening period, and be at a dangerously advanced state at the next test some 5 years or more later.

Therefore, it is clearly advantageous to employ the methods of the present invention which allow for simultaneous assaying of both preceding conditions such as HPV infection, as well as later stages such as abnormal cellular proliferation. In this way, according to the present invention, whenever a patient presents for testing, the disease should be detected whether it is in its infancy, or at a more critical advanced state.

Further, by combining the testing procedures according to the present invention, the doctor is presented with a better overall picture of the condition of the patient. It is possible to have only HPV infections, or to have an isolated advanced lesion, or to have numerous different abnormalities at different stages of development. By combining the tests according to the present invention, it is substantially easier to gain an appreciation of the condition of the patient, and to properly plan a course of appropriate treatment.

In addition, it is an advantage of the present invention that the necessity for complicated interpretation of histological samples is significantly alleviated. The methods of the present invention employ molecular tests which are easier to score than conventional smear tests which require a highly trained technician to judge the state of the cells by their appearance. Molecular tests according to the present invention may be scored in a simple positive or negative fashion, rarely requiring a difficult judgement to be made. Indeed, it is an advantage of the present invention that it lends itself to straightforward automation. It would be possible to score the tests using a fluorescent reader or similar device which could be programmed to read at two or more different wavelengths and therefore provide information as to the various possible states of the sample, as opposed to a single state as prior art methods provide.

It is envisaged that the standard method of detection may be modified. Antibody binding may be carried out while the cells are in suspension, with cells being spun down prior to analysis. This would improve the quality of the screen.

Considerable effort in diagnosis is aimed at automating screening methods. The use of antibodies or antigen-binding variants thereof for HPV or cell proliferation marker detection greatly facilitates this.

The invention will now be described by way of illustrative examples .

### EXAMPLE 1

### Preparation of Anti-E4 Monoclonal and Polyclonal Immunoglobulins

Although Mabs against HPV 16 E1\hat{ }E4 have been described previously (TVG401, 402, 403; Doorbar et al, 1992) these reagents recognise a single overlapping epitope at the major antigenic site of E4, and have been reported not to detect the protein in archival tissue biopsies (Doorbar et al, 1992).

Although these results suggest that E4 may not be a candidate for immunological detection of HPV, further antibodies are generated targeted at the N and C termini of HPV16 E4.

The generation of further Mabs by standard hybridoma technology results in the isolation of TVG404, an IgM which recognises an epitope at the very C-terminus of the protein.

To complement this reagent polyclonal antiserum to the N-terminus of the protein is raised against an N-terminal synthetic peptide (E4 N term). Polyclonal antibodies (to HPV16 and HPV63 E4 proteins) are prepared by immunisation of rabbits with maltose binding protein E4 fusion protein (MBP-E4). Antibody titres are monitored in ELISA using purified glutathione S transferase E4 fusion protein (GST-E4).

Antibodies to the N-terminus of the protein are raised against the synthetic peptide MADPAAATKYPLC after conjugation to thyroglobulin or keyhole limpet haemocyanin through its C-terminal cysteine residue. Conjugation is carried out using m-Maleimidobenzoyl-N-hydroxysuccinimide ester (MBS) as described by Green et al (1982).

Antibody specificities are confirmed by epitope mapping, as follows: the HPV 16 E4 protein is synthesised as a series of 85 overlapping octamers (single amino acid overlap) by solid phase fmoc chemistry using the SPOTS epitope mapping system (Genosys Biotechnologies, Cambridge, UK). Accuracy of synthesis is confirmed using the HPV16 E1\hat{ }E4 monoclonal TVG402 which binds the major antigenic site of the protein (Doorbar et al, 1992). Filters are regenerated as described by the manufacturers and antibody binding is visualised by ECL (Amersham, Little Chalfont, UK). Polyclonal serum is used at 1/250 dilution, purified Fabs at approximately 1 g/ml, and hybridoma supernatant at 1/10 dilution.

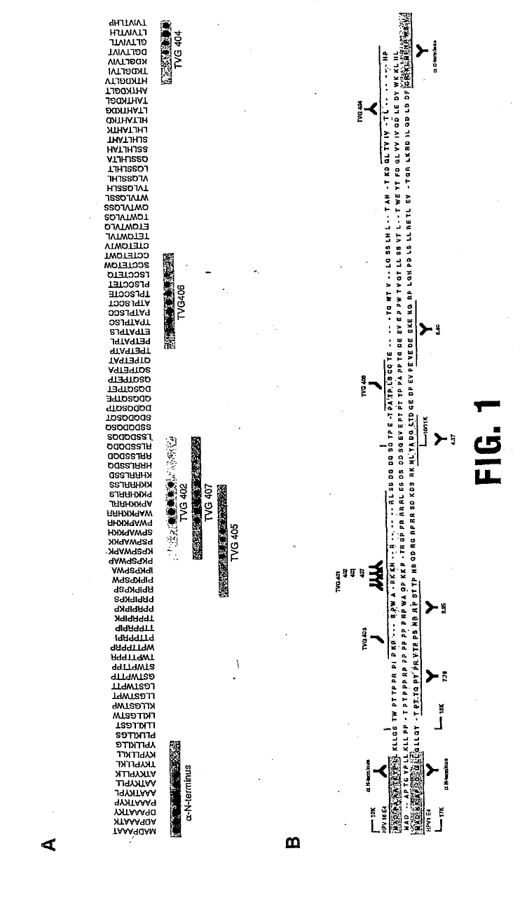

In FIG. 1A the sequences of the 85 overlapping E4 synthetic peptides are shown at the top of the figure, and the results of the epitope mapping experiments are shown below. The dark spots represent binding of the antibody to the synthetic peptide shown above it. Only the portion of the filter containing peptides which react with each antibody are shown.

In FIG. 1B, the locations of epitopes on the E1\hat{ }E4 amino acid sequence are summarised above the HPV16 sequence. Alignment with a consensus E4 sequence prepared by comparison of 70 putative E1\hat{ }E4 sequences (Doorbar and Myers, 1996b) is shown beneath the sequence of HPV16 E1\hat{ }E4, and the sequence of the HPV1 E1\hat{ }E4 protein is shown beneath this. The binding sites of the existing HPV 1 E1\hat{ }E4 Mabs (Doorbar et al, 1988) are shown beneath the HPV1 sequence. The proteolytic cleavage sites that give rise to the 16K and 10/11K gene products in the E1\hat{ }E4 protein of HPV1 (Doorbar et al, 1988; Roberts et al, 1994) are indicated beneath the HPV1 sequence allowing prediction of putative sites in the E1\hat{ }E4 sequence of HPV 16.

### EXAMPLE 2

### Preparation of Synthetic Immunoglobulins

Fabs are isolated from a synthetic antibody displayed on fd bacteriophage (Griffiths et al, 1994) as described below. Immunotubes (Life Technologies, Paisley, UK) are coated overnight at 4° C. with either MBP-E4 or GST-E4 at a concentration of 0.1 g/ml. These are subsequently blocked at 37° C. for 1 hour in PBS/2% marvel™ prior to incubation in the presence of 10¹¹ phage on a blood tube rotator (37° C. ). Unbound phage are poured off and tubes are washed 10× with PBS/0.1% Tween 20. Binders are eluted with 100 mM triethylamine pH 11.0 (1 ml) and immediately neutralised with 1M Tris (pH8.0) before being reintroduced into E. coli TG1 cells. The enriched library is grown up and the selection procedure repeated three more times.

Phage selections are carried out alternately against GST 16 E1\hat{ }E4 and MBP 16 E1\hat{ }E4 in order to prevent isolation of antibodies to MBP or GST protein, using a repertoire of 6.5×10¹⁰ (Griffiths et al, 1994). MBP 16 E4 is expressed at higher levels (>50 mg/litre of bacteria) than the GST fusion (approx. 5 mg/litre of bacteria) but, in any event, antibody isolation requires as little as 1 g of antigen (Hawkins et al, 1992). Phage displaying antibodies with affinity for E4 are identified by ELISA (against GST-E4, MBP-E4, GST and MBP), and activity is confirmed by phage western blotting. Immunoglobulin genes are transferred from the isolated phage into the bacterial expression vector pUC^{119.}His.myc (Griffiths et al 1994) and soluble Fabs are purified from the periplasmic space of induced bacteria by Nickel-NTA chromatography (Qiagen, Crawley UK). Antibody titres are established by ELISA.

After four rounds of selection, individual clones are examined for their ability to bind either E1\hat{ }E4, unfused GST or MBP, or bovine serum albumin (BSA). 47 clones (out of 100) are able to bind MBP 16 E1\hat{ }E4, of which 39 could also bind GST 16 E4. None of these clones interacted with BSA, GST or MBP. BstNI fingerprinting (Marks et al, 1992: Nissim et al, 1994) revealed three distinct Fabs amongst these clones, and their immunoglobulin genes are subcloned into the prokaryotic expression vector pUC119His.6myc to allow the production of soluble anti-E4 Fabs (Griffiths et al, 1994). Approximately 5 mg (per litre of bacteria) of anti-E4 Fab (TVG 405, 406 and 407) can be extracted from the periplasmic space of induced bacteria and all are found to specifically detect E1\hat{ }E4 by ELISA and western blotting. Fab TVG 407 binds an epitope which is identical to that recognised by the hybridoma-derived Mab, TVG 409 (FIG. 1). The remaining synthetic Fabs recognise novel epitopes upstream (TVG 405) or downstream (TVG 407) of this major antigenic region of E4 and the results are summarised in FIG. 1.

It is found that the amino acid sequence of the CDR3 loops of the TVG 405 and TVG 407 Fabs are as follows:

| | |
|---|---|
| TVG 405 | heavy chain CDR3 sequence: LLRGAFDY |
| | light chain CDR3 sequence: NSRDSSGGNAV |
| TVG 407 | heavy chain CDR3 sequence: LVQGSFDY |
| | light chain CDR3 sequence: QADSSTHV |

Measurement of Antibody Affinity

Affinities of synthetic (TVG405, TVG406 and TVG407), and hybridoma-derived Fabs (TVG402) are analysed by surface plasmon resonance using a BIAcore 2000 instrument (Pharmacia Biosensor, St. Albans, UK) as described by the manufacturer. MBP-E4 aggregates are dissociated under reducing conditions in 0.5% SDS, 1 mM β-mercaptoethanol, 50 mM Na₂CO₃/NaHCO₃ (pH 8.5) and biotinylated using NHS-LC-biotin (Sigma, St Louis, USA; 25 mg/ml in DMSO) at a biotin:protein molar ratio of 6:1 (Johnson et al, 1991). Monomeric MBP-E4 is recovered by FPLC chromatography using a Superdex S200 HR10/30 column run in 6M Urea/1 mM β-mercaptoethanol/PBS/0.2 mM EDTA (pH7 2), before being bound to a streptavidin-coated sensor chip and “refolded” in vitro in PBS/0.2 mM EDTA/0.1mg/ml protease-free BSA (Sigma). Fabs are isolated from purified TVG402 using an Immunopure Fab kit (Pierce, Rockford, USA), and monomeric preparations are obtained by FPLC gel chromatography (Superdex S200 HR10/30 column run in PBS/0.2 mM EDTA (pH 7.2)) Sensor chip surfaces are regenerated using 6M urea column buffer (described above). On and off rates are derived by non linear curve fitting using the proprietary ‘BIAanalysis’ software.

Binding activities are in the order of 20% of total protein levels for the bacterially-derived antibodies, and 50% for Fabs derived from hybridoma culture supernatant. The affinities of TVG405 and TVG402 are calculated from on- and off-rates obtained by non-linear curve fitting to sets of BIAcore binding curves.

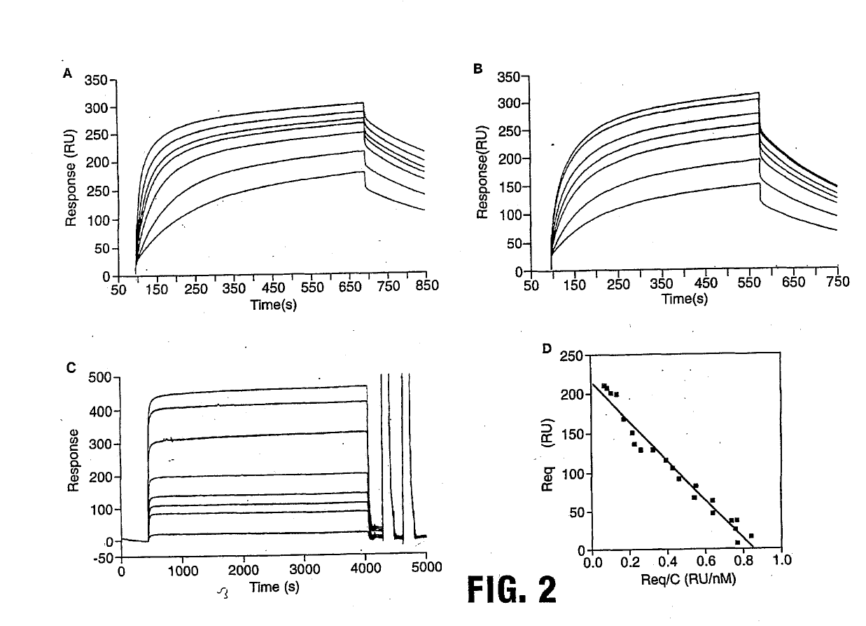

FIG. 2A shows an overlay of binding curves (sensograms) obtained after passing Fab TVG405 over a BIAcore chip coated with MBP-E4 fusion protein as described above. Fab concentrations range from 10 mM (lowest curve) to 300 nM (upper curve) through 5 intermediate dilutions. The extent of binding is indicated in resonance units on the X-axis, against time in seconds on the Y-axis. Purified Fab is injected at around 100 seconds and washing initiated at 700 seconds. The affinity (K_{d}) of TVG405 is calculated as between 0.3 and 1.25 nM by analysis of the association and dissociation curves using BIAevaluation software (Pharmacia, UK).

FIG. 2B shows an overlay of binding curves (as described above) for the hybridoma-derived Fab TVG402 over a concentration range 100 nM to 1 M. The K_{d} is estimated as 70 nM.

TVG405 has an association rate constant (kₒₙ) of 1.8×10⁶ M⁻¹.s⁻¹ and an off rate (k_{off}) of 2×10³ s⁻¹ indicating a molar dissociation constant (Kd) of approximately 1 nM. The best hybridoma-derived antibody—TVG402—has an affinity of only 70 nM, and had a kₒₙ of 4.2×10⁴ M⁻¹.s⁻¹ and a k_{off} value of 3×10³ s⁻¹. TVG 406 and 407 display rapid kinetics and are thus examined by Scatchard analysis of equilibrium binding data, as shown for TVG407.

FIG. 2C shows the equilibrium binding curve of Fab TVG407, which displays rapid kinetics. FIG. 2D shows Scatchard analysis of the data presented in FIG. 2C using BIAevaluation software. Equilibrium values are corrected for bulk refractive index changes by subtracting values from a surface blocked with biotin, from the values shown in FIG. 2C. In the plot shown the slope is −K_{d} and the Y-axis intercept is ‘Rₘₐₓ’, i.e. the binding level at saturation with Fab. The uncorrected K_{d} values for TVG407 and TVG406 are 250 nM and 140 nM which, when the activity of the Fab preparation is considered, indicates actual affinities of 50 nM and 28 nM.

TVG407 has an affinity (Kd) of 50 nm after correction for biological activity, and TVG406 has an affinity (Kd) of 28 nM. The amino acid sequence of the heavy and light chain CDR3 loops are established by DNA sequencing, further confirming that the three antibodies are distinct.

### EXAMPLE 3

### Preparation of Anti-E4 Peptides

A commercially available two-hybrid screening kit is purchased from ClonTech and employed for identifying naturally occurring E4-binding peptides, according to the instructions given by the manufacturer. A HeLa cDNA library, obtained from the same supplier, is screened. By this method, seven DNA sequences are isolated which encode E4-binding polypeptides, of which three are identified after sequencing.

The first peptide is ferritin.

The second peptide is a keratin filament binding protein, which has the sequence set forth in SEQ. ID. No. 2.

The third polypeptide is a novel polypeptide recognised as a member of the DEADbox family of proteins, which contain the characteristic sequence motif DEAD. The sequence of the third polypeptide is shown in SEQ. ID. No. 3.

In order to identify the site of interaction between these polypeptides and E4, a series of overlapping peptides of between 10 and 20 amino acids in length is generated by PCR and displayed on phage as described above. The binders are subsequently employed as screening agents to identify HPV 16 in mucosal lesions.

### EXAMPLE 4

### Preparation of Anti-E4 RNA Oligonucleotides

RNA oligonucleotides, known as aptamers, which are capable of specific binding to target molecules can be generated by selection procedures such as SELEX. The SELEX method involves selection of nucleic acid aptamers, single-stranded nucleic acids capable of binding to a desired target, from a library of oligonucleotides. Starting from a library of nucleic acids, preferably comprising a segment of randomised sequence, the SELEX method includes steps of contacting the library with the target under conditions favourable for binding, partitioning unbound nucleic acids from those nucleic acids which have bound specifically to target molecules, dissociating the nucleic acid-target complexes, amplfying the nucleic acids dissociated from the nucleic acid-target complexes to yield a ligand-enriched library of nucleic acids, then reiterating the steps of binding, partitioning, dissociating and amplifying through as many cycles as desired to yield highly specific, high affinity nucleic acid ligands to the target molecule.

DNA Oligonucleotide Library

DNA oligonucleotides 73 bases in length, having a randomised portion of 26 bases, are used for the development of an aptamer capable of binding E4. A library of synthetic RNA oligonucleotides having the following structure is prepared:

5′ CCTGTTGTGAGCCTCCTGTCGAA(26N)TTGAGCGTTTATTCTTGTCTCCC 3′

Where N stands for any possible base in the random region. The random region is generated by using a mixture of all four nucleotides (ratio 6:5:5:4, A:C:G:T, to allow for differences in coupling efficiency) during the synthesis of each nucleotide in that stretch of the oligonucleotide library. The resulting complexity is theoretically 4²⁶ molecules. The scale of synthesis (0.1 μmol) followed by gel purification yields 8.8 nmol which puts an absolute upper limit of approximately 5×10¹⁵ on the number of different molecules actually present.

PCR Amplification with a 5′ primer that introduces the recognition site for T7 RNA Polymerase (5′ TAATACGACTCACTATAGGGAGACAAGAATAAACGCTCAA 3′) and 3′primer (5′ GCCTGTTGTGAGCCTCCTGTCGAA 3′) results in the following template for transcription:

5′TAATAGCACTCACTATAGGGAGACAAGAATAAACGCTCAA (26N)
TTCGACAGGAGGCTCACAACAGGC 3′

The RNA transcript itself has the following sequence:

5′ GGGAGACAAGAAUAAACGCUCAA (26N)
UUCGACAGGAGGCUCACAACAGGC 3′

Anti-E4 aptamers are selected using a conventional SELEX procedure as described in U.S. Pat. No. 5,270,163. Each round consists of the following steps:

1) Selection. The RNA and E4 protein are mixed, incubated at 37° C. washed through a nitrocellulose filter, and RNA is eluted from the filters.

2) Amplification. The RNA eluted from filters is extended with AMV reverse transcriptase in the presence of 50 picomoles of 3′ primer in a 50 μl reaction under conditions described in Gauss et al. (1987). To the resulting cDNA synthesis 50 picomoles of 5′ primer is added and in a reaction volume of 100 μl and amplified with Taq DNA polymerase as described in Innis (1988) for 30 cycles.

3) Transcription. In vitro transcription is performed on the selected amplified templates as described in Milligan et al. (1987), after which DNaseI is added to remove the DNA template. The resultant selected RNA transcripts are then used in step 1 of the next round. Only one-twentieth of the products created at each step of the cycle are used in the subsequent cycles so that the history of the selection can be traced. The progress of the selection method is monitored by filter binding assays of labelled transcripts from each PCR reaction. After the fourth round of selection and amplification, the labelled selected RNA products produce binding to E4. The binders are used in the detection of HPV in cells derived from cervical smears.

### EXAMPLE 5

### Detection of HPV in Cutaneous and Mucosal Lesions

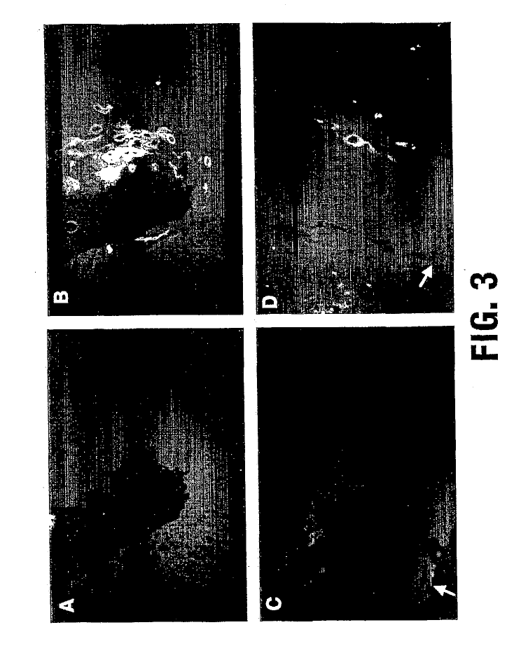

All the synthetic Fabs detect the HPV16 E1\hat{ }E4 protein in formalin fixed paraffin-embedded tissue, although TVG405 consistently show the highest level of staining (FIG. 3).

FIG. 3 illustrates the use of synthetic Fabs to localise HPV16 E4 protein in vivo by immunostaining of a low grade HPV16 CIN I with Fab NIP-C11 (Griffiths et al, 1994), which has no reactivity towards HPV16 E4 (FIG. 3A), and the E4-specific Fab TVG405 which is described here (FIGS. 3B, C, D). Fabs are detected using 9E10 as secondary antibody followed by anti-mouse FITC conjugate. E4 is detectable in the upper layers of the epidermis but at greatly varying levels between different lesions with often only a few positive cells being apparent (C, D). The position of the basal layer is arrowed in C and D. Magnification is 200×.

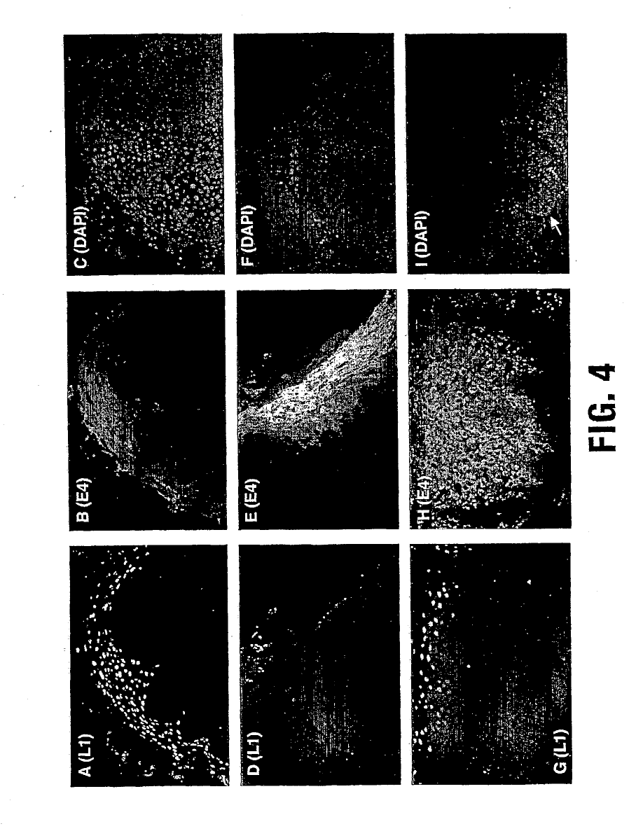

Epitope exposure by microwave treatment enhances the sensitivity of E4 detection, and even allows staining using TVG402 (Doorbar et al, 1992). The extent of E4 expression varies greatly between different lesions (8 HPV16-associated CIN1 biopsies are examined), ranging from expression only in rare cells scattered throughout the biopsy (FIG. 3), to widespread distribution throughout the most differentiated layers of the epidermis (FIG. 4), comparable to the distribution of E4 in cutaneous warts caused by HPV1 and HPV63 where the production of infections virions is also high (FIG. 4). In low grade cervical intraepithelial neoplasia (CIN 1) caused by HPV16, E4 and L1 levels are also found to correlate closely, although expression of the two proteins is not coincident (as previously suggested (Brown et al, 1994). E4 expression precedes the synthesis of the major capsid protein by several cell layers (as revealed by double staining, see FIG. 4) and in high grade cervical lesions (CIN 2/CIN 3) E4 is often abundant, even though the expression of L1 is no longer supported (FIG. 4). This time delay between the commencement of E4 synthesis and the assembly of infectious virions is most apparent in HPV63, where E4 expression coincided with migration of an infected basal cell into the parabasal layers, while expression of L1 is restricted to a narrow strip of cells in the upper granular layer.

FIG. 4 demonstrates that synthesis of E4 is not directly linked to the expression of capsid proteins in high and low grade HPV16 lesions, and benign warts. FIG. 4 shows the results of triple staining using anti L1 antisera (FIGS. 4A, D, G), HPV16 E4 Fab TVG405 (FIGS. 4B and 4E), polyclonal antisera to HPV63 E4 (FIG. 4H), and with DAPI (FIGS. 4C, F, I). A, B and C represent a low grade HPV16-induced lesion (CIN I). D, E and F represent a high grade HPV16-induced lesion (CIN II/II). G, H and I represent a section through a verruca caused by HPV63. In all cases E4 expression precedes L1 expression although by only a few cell layers in CIN I (A, B). In the CIN II/III we assume that terminal differentiation is insufficient to support synthesis of virion structural proteins (D) although E4 expression is abundant (E). The contrast between the onset of E4 expression and the detection of virus structural proteins is most apparent in cutaneous verrucas caused by HPV63 (G, H). The basal layer is indicated by an arrow on the DAPI-stained images. Magnification is 100×.

Onset of Vegetative Viral DNA Replication and Expression of E4 Coincide

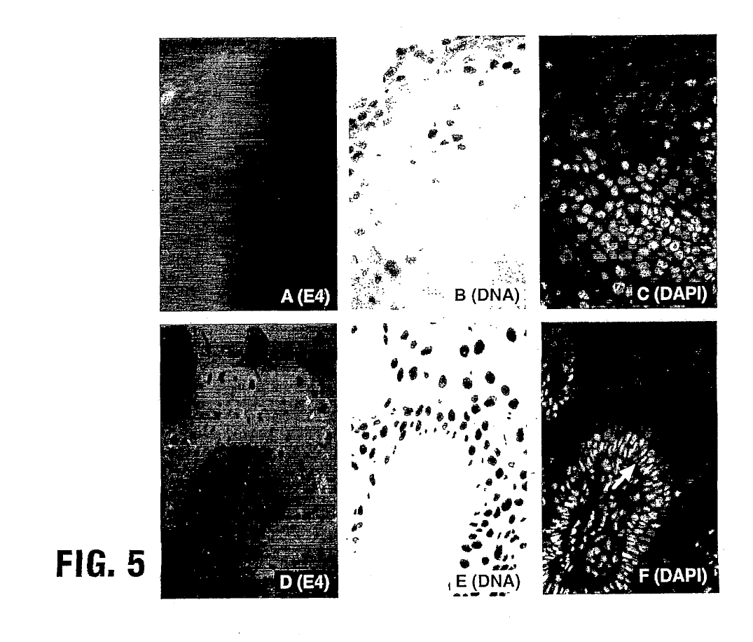

Vegetative viral DNA replication is found to begin in cells of the mid spinous layer and to correlate exactly with the onset of E4 expression (FIG. 5).

FIG. 5 demonstrates that onset of vegetative viral DNA replication coincides with E4 expression in low grade HPV 16 lesions and in benign cutaneous warts. The figure shows triple staining using the HPV16 E4 antibodies TVG402, 405 and 406 (FIG. 5A) and HPV1 E4 antibodies 4.37 and 9.95 (FIG. 5D), biotinylated DNA probe (FIG. 5B—HPV16, FIG. 5E—HPV1), or DAPI (FIGS. 5C and F). A, B and C represent a section through an HPV16-induced CIN I, and D, E and F represent a section through an HPV1-induced verruca. In the HPV16 CIN I, vegetative viral DNA replication and E4 synthesis correlate in the mid to upper layers of the epidermis (A, B). In cutaneous lesions the two events are initiated as soon as the infected cell leaves the basal layer (D, E). Basal cells are illustrated in the DAPI counterstained image (F). Magnification is 200×.

In HPV 1-induced warts vegetative viral DNA replication and E4 synthesis commence much earlier, and are evident immediately after the infected basal cell migrates into the superficial layers (FIG. 5). Only a proportion of the differentiating cells are permissive for vegetative viral DNA replication, and only in these cells is E4 detectable. Neighbouring cells showed neither late gene expression nor vegetative viral DNA replication, suggesting that onset of the two events is closely linked. Although the sensitivity of DNA and E4 detection is not established, these ‘normal’ cells are likely to be either non-permissive for viral replication or be uninfected. This precise correlation between E4 expression and the onset of vegetative viral DNA replication is also seen in cutaneous warts caused by HPV63 and 65, and in common warts caused by HPV2.

Cells Undergoing Late Gene Expression Show an Abnormal Pattern of Terminal Differentiation when Compared to Non-Permissive or Uninfected Cells

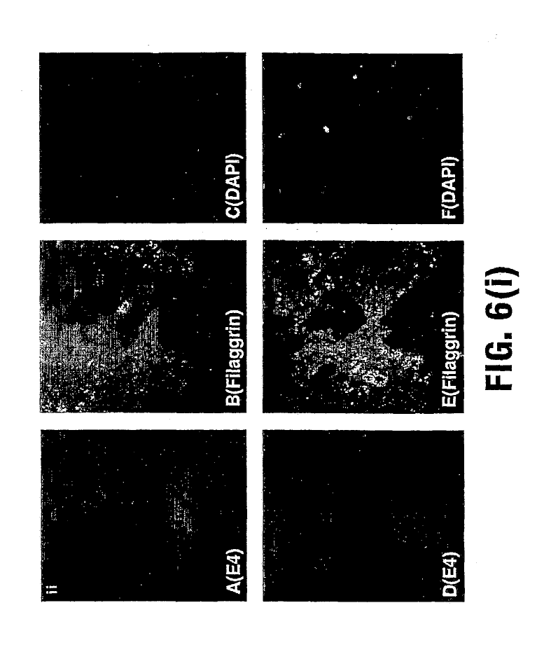

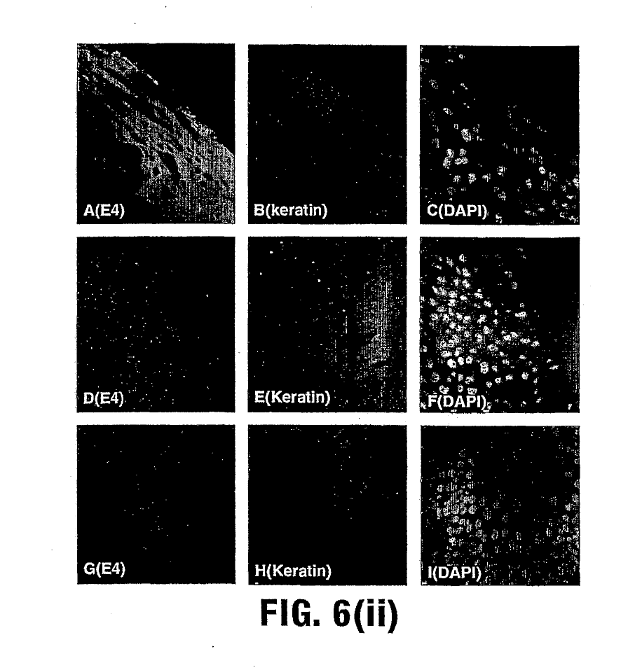

Cells supporting the late stages of HPV infection can thus be identified by immunostaining with Fab TVG405 (for HPV16) Mab 4.37 (for HPV1) or polyclonal antisera to E4 (HPV63). In warts caused by HPV1, E4-positive cells lack detectable levels of filaggrin or involucrin (FIG. 6(i)). Non-permissive (or uninfected) cells in the same lesion which show neither E4 expression nor vegetative viral DNA replication, express filaggrin and loricrin at levels indistinguishable from those in the surrounding epidermis. Correlation of E4 synthesis with the differentiation-specific keratins K4 and K13 reveals an identical pattern of inhibition. The intensity of K4 and K13 staining is always lower in E4-positive cells than in neighbouring cells that are not expressing E4 (FIG. 6(ii)). K5 and 14, which are present in the basal and lower parabasal cells, are unaffected. This interference with the detection of expression differentiation-specific keratins (K1 and K10 in cutaneous skin) is also apparent in cutaneous warts caused by HPVI (FIG. 6(ii)) but is not evident in warts caused by HPV63 (FIG. 6(ii)). The E4 protein of HPV63 is most closely related to that of HPV1.

FIG. 6 illustrates that productive infection interferes with normal epithelial terminal differentiation in low grade HPV16 lesions and in benign cutaneous warts. FIG. 6(i) (keratin expression) shows triple staining using the HPV16 E4 Fabs TVG405/TVG406 (FIG. 6(i)A), HPV 1 E4 monoclonals 4.37/9.95 (D), and HPV63 E4 polyclonal antibodies (G), in conjunction with antibodies to the differentiation-specific mucosal keratins 4 and 13 (B) or cutaneous keratins 1 and 10 (E, H). FIGS. 6(i) C, F and I show the DAPI counter stain. A, B and C represent a section through a HPV16-induced CIN I. D, E and F show a section through the edge of an HPV1-induced verruca, while FIGS. 6(i) G, H and I show a section through an HPV63-induced wart. In HPV16. and HPV1-induced lesions, differentiation-specific keratins are less apparent in E4-positive cells than in neighbouring cells (A, B, D, E) although this is not the case with HPV63 (G, H). Nuclear degeneration (visualised by DAPI counter staining) is retarded in E4-expressing cells (A, C, D, F). Magnification is 200×.

FIG. 6(ii) relates to filaggrin expression. The figure shows triple staining, as described above, except that FIGS. 6(ii) B and E show filaggrin staining. E4 staining is shown in FIGS. 6(ii) A and D, and DAPI counter staining is shown in FIGS. 6(ii) C and F. A, B and C represent the edge of an HPV63-induced wart where normal skin (left hand side of figure) meets the benign tumour (right hand side of figure). D, E and F show the granular layer of an HPV1-induced wart. E4-positive cells do not express detectable levels of the differentiation-specific marker filaggrin, and show marked nuclear preservation when compared to neighbouring uninfected or non-permissive cells. Magnification is 200×.

The Intracellular Distribution of the HPV16 E4 Proteins is Distinct from the Distribution of E4 in Cutaneous Lesions Caused by HPV1 and HPV63.

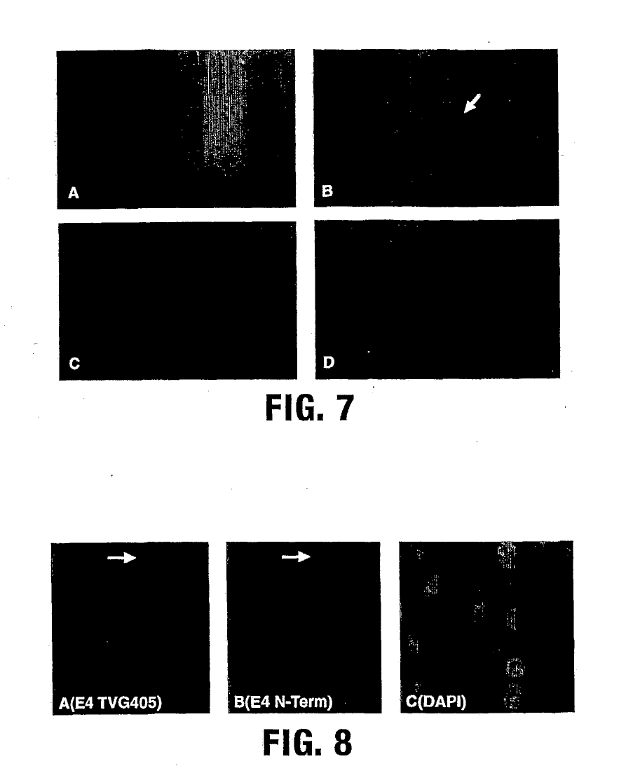

The E1\hat{ }E4 protein of HPV1 is predominantly cytoplasmic and assembles into inclusions that coalesce and increase in size as the cell migrates towards the surface of the skin. The E1\hat{ }E4 protein of HPV63 is found to have a fibrous and granular distribution. By contrast, HPV16 E4 had a filamentous and perinuclear distribution in cells of the lower epidermal layers (FIG. 7), and assembled into prominent structures only in the more differentiated cell layers. These ‘inclusions’ are always found singly per cell (c.f. multiple inclusions found in most cutaneous lesions), are located adjacent to the nucleus, and are nearly always detected on the side of the nucleus closest to the surface of the epidermis. Although similar in appearance to the E4/intermediate filament bundles which form after expression of the HPV 16 E1\hat{ }E4 protein in epithelial cells in vitro, we have not detected the presence of keratins in these structures in vivo. Antibodies to the very N-terminus of HPV16 E1\hat{ }E4 stained the structures much less readily than antibodies to C-terminal epitopes (TVG 404, TVG405, TVG406) suggesting that the N-terminal region maybe either hidden or lost.

FIG. 7 shows the association of the HPV16 E4 proteins with perinuclear bundles and filamentous structure in vivo, in particular the detection of HPV16. E4 proteins in the upper layers (FIGS. 7A, B) and lower layers (FIGS. 7C, D) of a HPV16 CIN I using a mixture of Fabs TVG405 and TVG406. In the upper layers E4 is diffuse throughout the cytoplasm but with a prominent perinuclear pattern. Concentration of E4 into perinuclear bundles (arrowed in FIG. 7B) is apparent in these cells. In the lower layers, E4 has a predominantly perinuclear and filamentous appearance (FIGS. 7C, D), but is not concentrated into perinuclear bundles. Magnification for FIGS. 7A and C is 200×; that for B and D is 400×.

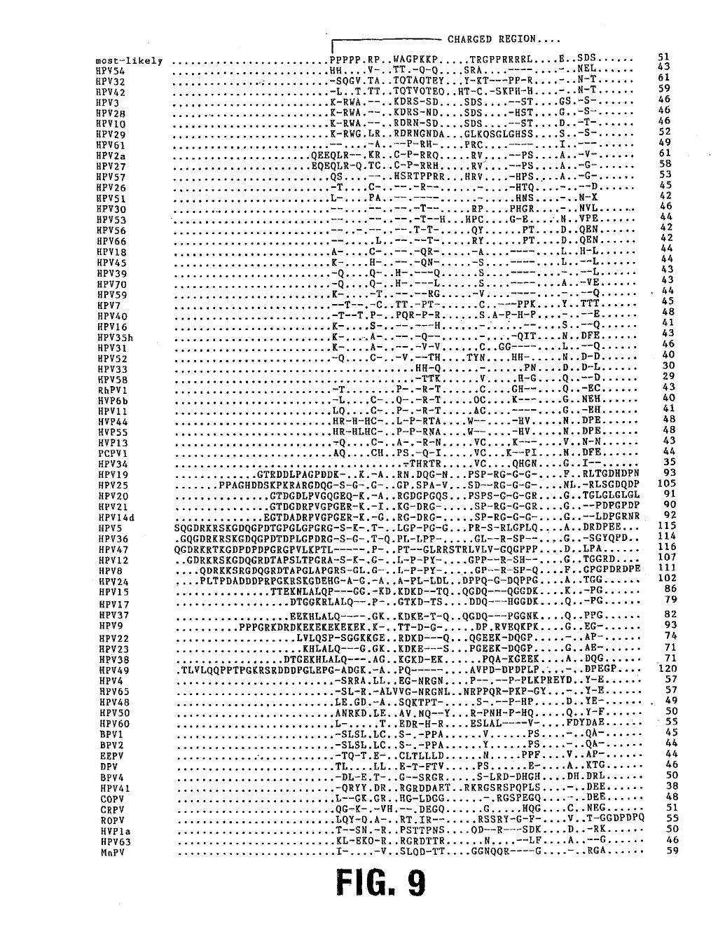

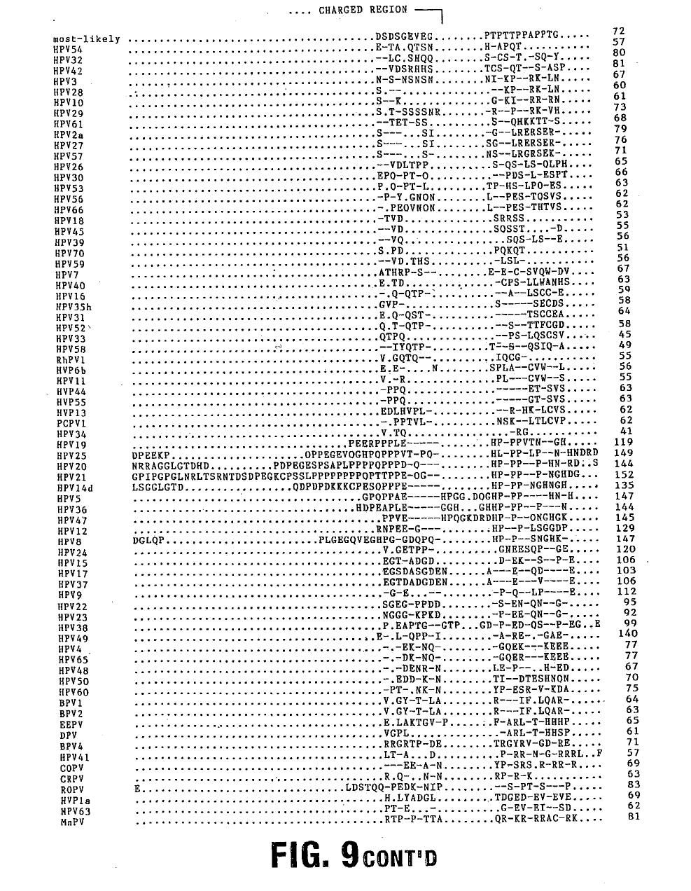

Confocal imaging revealed the N-terminal antibodies to localise primarily to the edge of the E4 structures while anti C-terminal staining is strongest in the centre (data not shown). When compared to the distribution seen with TVG405 and TVG406, the anti N-terminal reagent revealed HPV16 E1\hat{ }E4 to have a more diffuse distribution in the cell (FIG. 8).

No significant difference is apparent between the staining pattern of TVG405, 406, 407 and the C-terminal antibody.

FIG. 8 provides evidence for processing of the HPV16 E4 proteins in vivo and shows triple staining in the upper layers of a HPV16 CIN using HPV16 E4 Fab TVG406 which recognises an epitope in the C-terminal half of the E4 protein (FIG. 8A), an antibody to the N-terminal 12 amino acids of the HPV16 E1\hat{ }E4 protein (FIG. 8B) and DAPI (FIG. 8C). TVG402, 403, 404, 405 and 407 produced staining patterns that are not significantly different from that of TVG 406. Anti N-terminal antibodies did not effectively stain the perinuclear bundles (8B) which are evident with TVG406 (arrowed in 8A) suggesting that as in HPV1, different forms of the protein have different intracellular locations. Magnification is 400×.

### EXAMPLE 6

### Detection of HPV in Cells Isolated from Cervical Lesions

Slides suitable for imaging of cells derived from cervical smears stained using anti-E4 antibodies are prepared by the method set forth in U.S. Pat. No. 5,346,831. Cells are isolated from a patient according to conventional procedures and dissolved in 10 ml alcohol/saline buffer. The sample is prepared for centrifugation by disaggregating the clumps or clusters of cells in the sample vial by vortexing. After disaggregation, the sample is drained completely and layered over a density gradient in a 12 ml conical tube, wherein the density gradient is formed with a plasma expander material comprising 6% betastarch solution, and 0.9% physiological saline, also known by the tradename “Hespan” made by NPBI, Emmer-Compascuum, the Netherlands.

12 ml conical tubes containing density gradient and sample cells are placed into centrifuge buckets, balance and centrifuged for 5 minutes, at a force of about 600G. The liquid is then aspirated down to the 5 ml mark on the conical tube. The centrifuge buckets are removed and the 12 ml conical tube centrifuged with remaining liquid for 10 minutes, at 800G. The tubes are emptied of supernatant, tapping lightly 2 or 3 times at a 45 degree angle. The tubes now contain packed cells of varying volumes. Upon mixing to homogeneity, the pellets generally contain the same concentration of cells per unit volume of liquid.

50 μl of deionised H₂O is added, and the sample mixed by syringing 5 times through a 0.042 inch tip. Upon completion of mixing, 150 μl of sample followed by 500 μl of deionised H₂O is dispensed into a sedimentation vessel attached to a slide which has been conventionally coated with Poly-L lysine (1% Sigma). The transferred sample is allowed to settle within the vessel for approximately 10 minutes. The excess sample is aspirated off and the chamber rinsed with 2 ml deionised H₂O two times (aspirating between each addition).

FITC-labelled Fabs are then applied to the cells according to known procedures and the binding visualised by fluorescence microscopy.

### EXAMPLE 7

### Double Staining of Sections with Anti-MCM and Anti-HPV Antibodies

Protocols

Antigen Exposure by Microwaving followed by Fluorescent Immunostaining

Paraffin embedded sections were de-waxed in xylene (4×5 min), hydrated in IMS (3×5 min) and rinsed in de-ionised water for 2×5 min. Sections were microwaved on high power (800W) for 15 min in 500 ml antigen retrieval buffer (10 mM citric acid, pH 6.0), and allowed to cool in the buffer for 20 min. After washing twice for 5 min in PBS, slides were blocked in PBS, 10% serum, 1% BSA (v/v) for 2 hours at room temperature. Primary antibodies were diluted in PBS, 1% B SA, applied to the sections and incubated overnight at 4° C. in a humidified chamber. Stringent washes in PBS, 0.05% Tween 20 were carried out (4×5 min) followed by a rinse in PBS. Biotin labelled secondary antibodies, HPV 16E4 (Alexa 488-conjugated) and a nuclear counterstain, diluted in 1% BSA/PBS, were incubated on the section for 2 hours at room temperature. Following washes in PBS (3×5 min), the biotin signal was amplified with an SABC-AP kit (DAKO), followed by 3×5 min PBS washes. The Alkaline phosphatase signals were detected by a 10 min incubation with Fast Red. The reaction was halted after 10 min by rinsing in water. The samples were mounted in citifluor mounting medium (Agar Scientific) and viewed under fluorescent light with a Nikon Labophot II microscope. Digital images were captured with a SenSys monochrome camera and IPLab imaging software (Roper Scientific).

Antigen Exposure by Pressure Cooking followed by Enzymatic Detection

Slides were de-waxed, pressure cooked and blocked as above. Slides were incubated overnight at 4° C. with DIG labelled HPV 16 E4 (TVG405) and a mouse monoclonal MCM2 antibody, diluted 1:50 and 1:15 respectively in TBS, 1% BSA (w/v), 0.1% triton (v/v). After washing in TBS the slides were incubated with antiDIG-AP secondary antibody diluted 1:50 and anti-mouse biotin diluted 1:200 in TBS containing 1% BSA, for 1 hour at room temperature. After 3×5 min washes in TBS the MCM signal was amplified with an SABC-HRP kit (DAKO), followed by 3×5 min TBS washes. Signals were detected with DAB (diminobenzidine) and Fast Red. The reaction was halted by rinsing in water and a light haemotoxylin counter-stain was applied. Alternatively the E4 was detected with anti-DIG-HRP and DAB, and the MCM was amplified with an SABC-AP kit and Fast Red detection. Sections were dehydrated through graded alcohols and mounted in DPX mounting medium. Brightfield images were captured with Nikon Eclipse 600 microscope with a Nikon Lucia imaging camera and associated software.

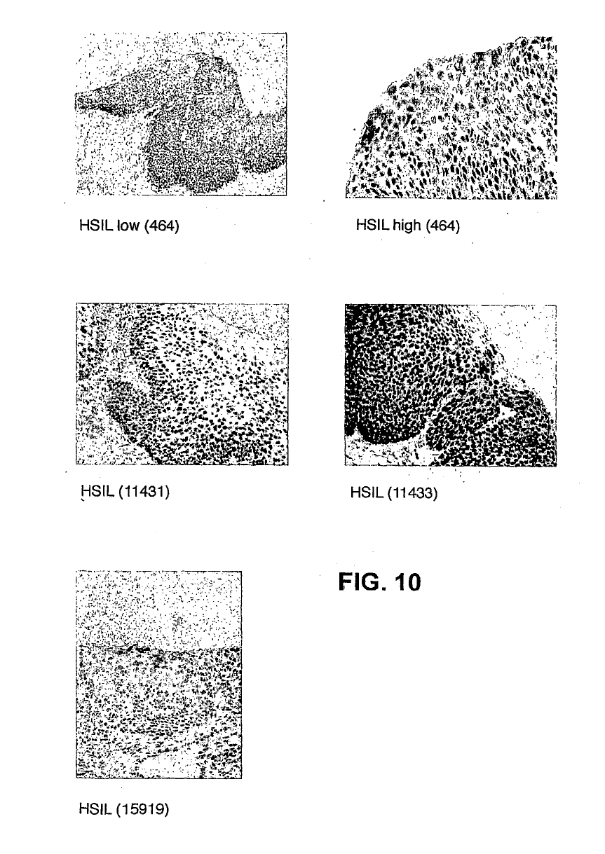

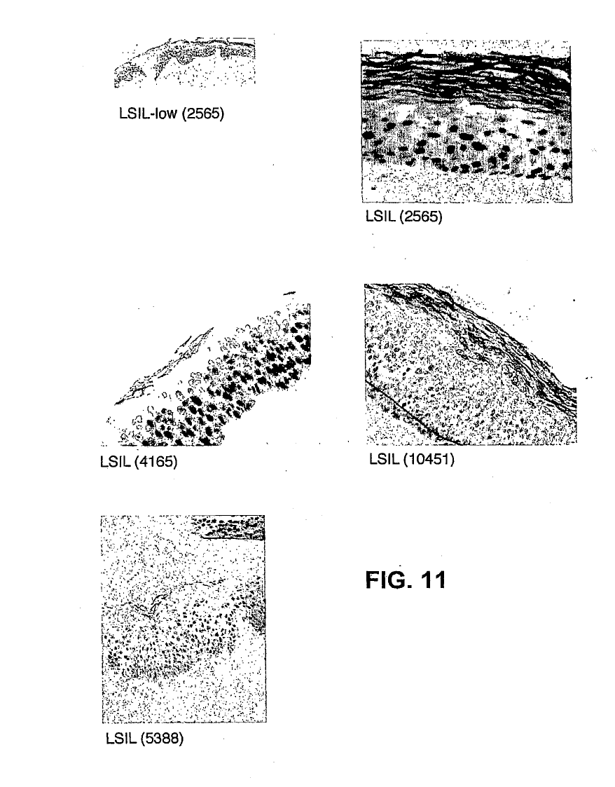

Double staining is carried out in HSIL and LSIL as described; the results are shown in FIGS. 10 and 11.

MCM staining is usually abundant in the surface layers of HSIL whereas expression of the E4 protein has a sporadic distribution. This is in agreement with the previous report by Williams et al. (PNAS 95, 1998) who showed that in HSIL, 95% of surface cells are positive for MCM staining. There has been no previous investigation of the E4/MCM colocalisation.

Although Williams et al reported that MCM staining was apparent in LSIL (53% of surface cells showing staining, PNAS 95 (1998) p14935), we have found that in regions that are positive for E4, MCM is usually absent from the surface layers (see 5388, 10451, 4165 and 2565). Some LSIL (e.g. see LSIL-low (2565)) which stain positive for E4 appear to lack any surface MCM staining. In the examples shown, E4 staining is widespread at the surface whereas MCM staining is absent.

From the data presented here, cervical scrapes taken from HSIL will show high levels of MCM staining but show only low levels of E4 expression. Moreover, the data suggest that E4 antibodies should allow detection of LSILs which can not be detected using antibodies to MCM proteins.

When the data presented in HSIL E4/MCM and LSIL E4/MCM are combined an additional conclusion can be made. The relative numbers of E4-positive and MCM-positive cells in the surface layers indicates the severity of disease. High numbers of MCM-positive cells and low numbers of E4-positive cells indicates the presence of HSIL. High levels of E4-positive cells and low numbers of MCM-positive cells in the surface layers is typical of LSIL. This type of analysis may be done using a cell sorter following double staining using antibodies to E4 and MCM.

Antibodies to MCM2, MCM5 and MCM7 gave essentially the same patterns for both HSIL and LSIL.

### EXAMPLE 8

### Double Staining of Normal Cervical Epithelium

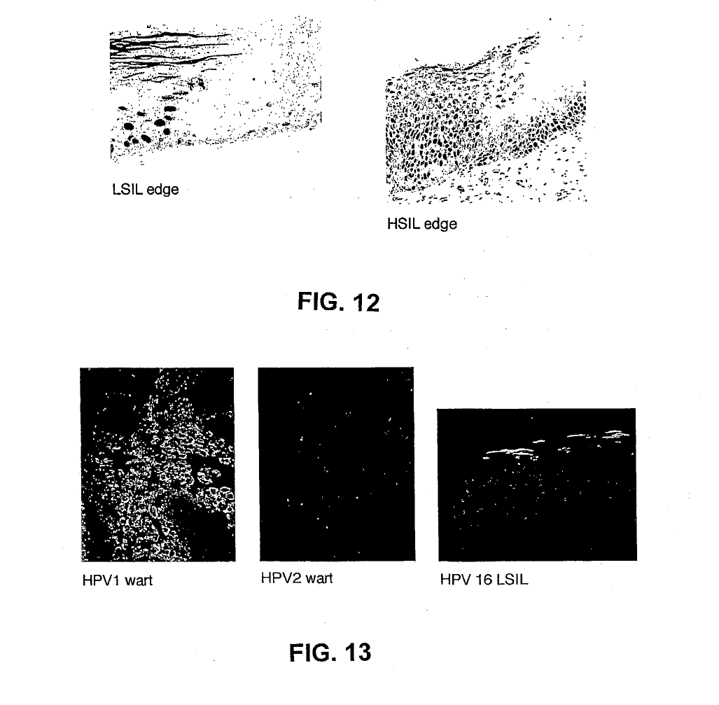

FIG. 12 shows double staining, performed as in Example 7, for normal cervical epithelium. Neither E4 nor MCM are present in the surface layers of normal cervical epithelium, emphasising the value of these antibodies for the detection of cervical abnormalities. The expression pattern of both proteins changes in regions of neoplasia and both E4 or MCM can be found at the surface.

### EXAMPLE 9

### Double Staining for E4/MCM in Other HPV Types

FIG. 13 shows double staining results obtained with HPV1, HPV2 and HPV 16 warts. The HPV E7 protein acts by binding to the cellular Rb gene product. In its unphosphorylated state, Rb normally prevents cell cycle progression by binding to the transcription factor E2F. During progression through G1, the levels of cyclin D kinase rise in response to growth factors acting externally on the cell. This leads to phosphorylation of Rb and release of E2F. E2F stimulates the synthesis of the proteins necessary for S-phase entry (reviewed in Virology edited by Fields, B. N., Knipe, D. M., Howley, P. M., published by Lippincott-Raven, Philadelphia, USA). Proteins involved in cellular DNA replication such as MCM, cdc6, PCNA and cyclin A are amongst these E2F-induced gene products (see Harbour & Dean, Nat Cell Biol 2000 April; 2(4):E65- 7).

In normal differentiating epithelial cells, cell cycle progression does not occur as the cells are committed to terminal differentiation. Neither S-phase markers nor HPV proteins are expressed in the differentiating cells of normal cervical epithelium. This is apparent from the images presented in Example 8.

In warts caused by HPV 1 and 2, and in E4-expressing LSIL caused by HPV 16, MCM is present in the lower layers of the epidermis. Based on our knowledge of HPV protein function this is predicted to be a result of E7 expression. The HPV E7 protein binds to Rb and displaces E2F which ultimately leads to entry into S-phase. Thus cellular genes which are activated by E7 can be considered as surrogate markers for the presence of the early viral genes. These genes are expressed from the p97 ‘early’ promoter in HPV16. In productive infections caused by HPV1, HPV2 and HPV16, the MCM proteins do not reach the surface of the epithelium. We predict that this is a common pattern which is likely to extend to all HPV types.

Vegetative viral DNA replication coincides with the first appearance of E4 (Doorbar et al., Virology 238 p40-52). E4 is expressed from the major late promoter, which in HPV16 is at p671. Thus the appearance of E4 marks the onset of the late stage of the virus life cycle. In lesions caused by HPV1, HPV2 and HPV16, the presence of E4 persists into the surface layers. Our recent work has shown that E4 can arrest the cell cycle in G2. In all three HPV types studied there is an overlap between the first appearance of E4 and the loss of staining for MCM. This observation leads us to suggest that these double positive cells are being pushed into S-phase by E7 but are unable to enter mitosis as a result of E4 expression. Such a situation is likely to be conducive for high level replication of the viral genome. This theory is compatible with all the experimental data gathered to date.

Thus we can summarise by stating that there are four types of cell:

1. The presence of MCM-positive cells at the surface can be regarded as a sign that productive infection is not being completed and that the virus is producing an abortive infection. During productive infection, MCM+/E4− cells are found only in the lower layers. Such cells are abundant in the surface layers only in HSIL and cancers.

2. MCM+/E4+ double positive cells are found in the intermediate layers in normal productive infections (i.e. in the MCM/E4 overlap region). Identification of these cells at the surface may indicate a lower grade lesion.

3. Cells that are +ve for E4 only (MCM−/E4+) and which lack S-phase markers altogether cannot be supporting vegetative viral replication as the proteins necessary for viral replication are not present. The presence of these cells at the surface is characteristic of lesions which have initiated productive infection. Productive infections can also be categorised as LSIL.

4. L1 expression follows E4. The presence of L1+/E4+/MCM− cells in a smear could be used to highlight a lower grade of lesion (HPV I) in which the life cycle is being completed. This has not been investigated.

Lesions caused by HPV 1 and HPV2 which do not progress to cancer, do not show these great variations. All cells at the surface are MCM−/E4+ and some express both E4 and L1 L1+/E4+/MCM−. Cells at the surface showing MCM+/E4+ and MCM+/E4− phenotypes would suggest the presence of an abortive infection such as HSIL.

These patterns are only seen in lesions such as those caused by HPV 16, which are progressing towards cancer. Lesions which are supporting the full HPV life cycle such as those caused by HPV1 and HPV2 have only L1+/E4+/MCM− and E4+/MCM− cells at their surface.

### EXAMPLE 9

### Other E2F-Induced Proteins can be Substituted for MCM

Most cervical abnormalities result from infection with a high risk papillomavirus. E7 is the viral protein which mediates cell proliferation. Surrogate markers of E7 expression such as MCM are present in all cervical neoplasias, but are confined to the lower epithelial layers in productive infections. Although MCM appear good markers of S-phase entry, there is no obvious reason why other cellular proteins induced by E2F could not be used in the same way. PCNA is essential for viral DNA replication and is also regulated by E2F (Hingorani & O'Donnell, Curr Biol 2000 Jan. 13;10(1):R25-9). PCNA has been widely used as a marker of proliferating cells. Antibodies to Ki67 have also been used for this purpose.

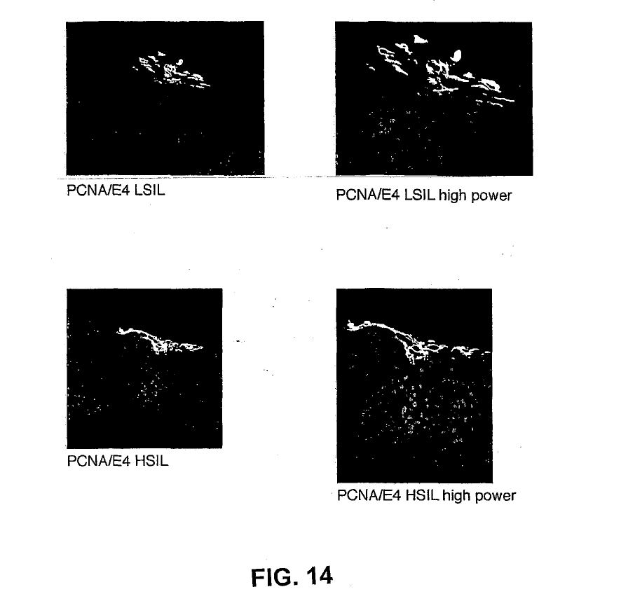

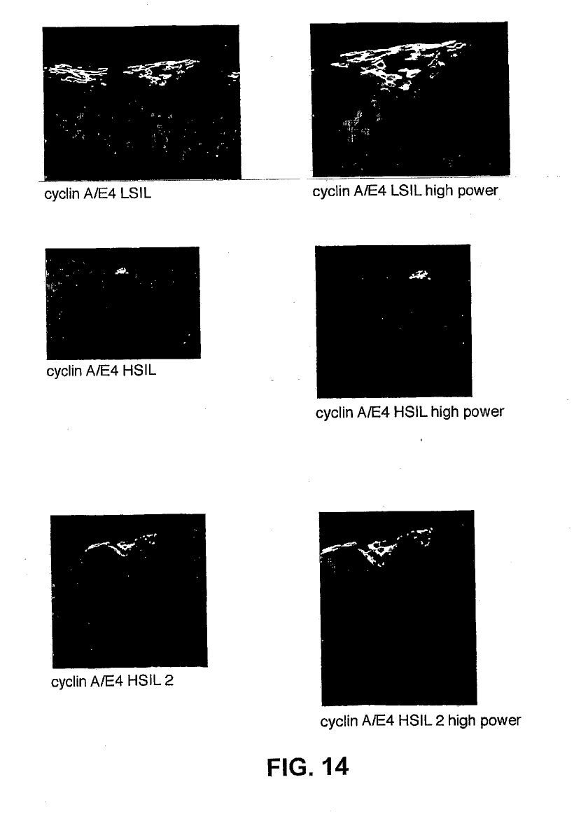

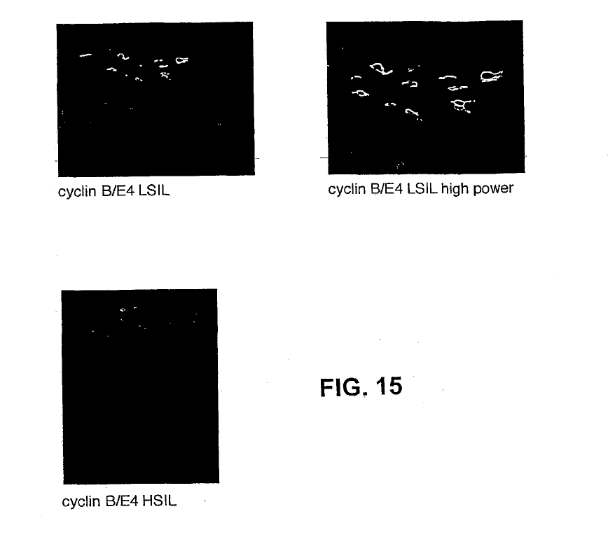

The images shown in FIG. 14 demonstrate that double stains using antibodies to PCNA and E4 are broadly similar to those produced using antibodies to E4 and MCM. During productive infection, PCNA is expressed in the lower epithelial layers but overlaps the region in which E4 is found. PCNA is present in the surface layers in HSIL.

We have also looked at the distribution of cyclin A which is also induced by E2F. Cyclin A is a kinase subunit that is essential for progression through S-phase. In HSIL, cyclin A extends into the surface layers. Double stains using antibodies to E4 and cyclin A reveal a region of overlap between the sites of expression of the two proteins. In most low grade lesions, cyclin A is confined to the lower epithelial layers whilst E4 is abundant at the surface.

Proteins such as PCNA and cyclin A thus have a similar overall distribution to MCM in both LSIL and HSIL. PCNA/E4 staining or cyclin A/E4 staining may thus be used for diagnosis in the same way as has been shown here for E4 and MCM. Although a comparison of the sensitivity of these approaches has not been carried out, PCNA appears to detect S-phase cells with a sensitivity that is similar to that of MCM. Other E2F-induced genes could be assessed in the same way.

### EXAMPLE 10

### Other Markers of Cell Proliferation can be Substituted for E2F Induced Genes

Cyclin B is a marker of G2 (Ohi et al., Curr Opin Cell Biol 1999 April;11(2):267-73). As shown in FIG. 15, cyclin B staining extends into the surface layers in HSIL. In LSIL in which E4 is expressed, these proteins are confined to the lower epithelial layers. As with PCNA, cyclin A and MCM, the regions which show cyclin B staining overlap the regions in which E4 can be detected in LSIL. Cyclin B does not extend into the surface layers in such lesions. Other cellular proteins which are induced by the virus may be used in conjunction with E4.

p16 is a cell cycle inhibitor of the INK family. This protein is normally upregulated in response to unscheduled entry into S-phase. p16 binds to cyclinD/cdk4 and cyclinD/cdk6 and inhibits its function. Cyclin D complexes normally phosphorylate Rb allowing release of E2F and entry into S phase. This regulation is bypassed by the HPV E7 protein. E7 binds directly to Rb and causes release of E2F irrespective of the presence of cyclinD. Thus in high risk HPV infections, p16 is abundant (Sano et al., Am J Pathol 1998 Dec;153(6):1741-8). Although p16 is not a proliferation marker it appears to be a useful marker of HPV early gene activity.

E4 staining may thus be combined with other markers of virus activity such as cyclin B and p16. These markers may be useful when used in conjunction with E4 for the diagnosis of cervical neoplasia.

References

Andrews & DiMaio, (1993) J. Virol. 67, 7705-7710.

Barksdale & Baker (1995). J. Virol 69, 6553-6556.

Beyer-Finkler et al, (1990) Med. Microbiol. Immunol. 179, 185-192.

Breitburd et al, (1987) In “Cancer Cells 5”, pp. 115-122. cold Spring Harbor Laboratory press, Cold Spring Harbor, N.Y.

Brown et al, (1994) Virology 201, 46-54.

Chow & Broker (1994), Intervirology 37, 150-158.

Chow et al, (1987) J. Virol. 61, 2581-2588.

Croissant et al, (1985) Clin. Dermatol. 3(4), 43-55.

Crum et al, (1990) Virology 178, 238-246.

Dietrich-Goetz et al, (1997) Proc. Natl. Acad. Sci. USA 94, 163-168.

Doorbar, J. (1996). The E4 proteins and their role in the viral life cycle. In “Papillomavirus Reviews: Current Research on Papillomaviruses” (C. Lacey, Ed.), pp. 31-38. Leeds Medical Information, Leeds University Press, Leeds.

Doorbar et al, (1989). EMBO J. 5(2), 355-362.

Doorbar et al, (1989). Virology 172, 51-62.

Doorbar et al, (1992). 187, 353-359.

Doorbar et al, (1991) Nature 352, 824-827.

Doorbar et al, (1988). EMBO J. 73, 825-833.

Doorbar & Gallimore (1987). J. Virol 61, 2793-2977.

Doorbar et al, (1996a). Virology 218, 114-126.

Doorbar & Myers (1996b). The E4 protein. In “Human Papillomaviruses 1996” (G. Myers, H. Delius, J. Icenogel, H.-U. Bernard, C. Baker, A. Halpern, and C. Wheeler, Eds), Vol. m, pp. 58-80. Los Alamos National Laboratory, Los Alamos, N.M., USA.

Egawa (1994) Brit. J. Dermatol. 130, 158-166.

Egawa et al, (1993a). Virology 194, 51-62.

Egawa et al, (1993b). Brit. J. Dermatol. 128, 271-276.

Frattini et al, (1996). Proc. Natl. Acad. Sci. USA. 93(7), 3062-3067.

Furth & Baker (1991). J. Virol. 65, 5806-5812.

Furth et al, (1994). Mol. Cell Biol. 14(8), 5278-5289.

Grand et al, (1989). Virology 170, 201-213.

Green et al, (1982). Cell 28, 477-487.

Griffiths et al, (1994). EMBO J.: 13,3245-3260.

Hawkins et al, (1992). J. Mol. Biol. 226, 889-896.

Hudson et al, (1992). Hybridoma 11(3), 367-378.

Hummel et al, (1995). J. Virol. 69, 3381-3388.

Jareborg & Burnett (1991). J. Gen. Virol. 72, 2269-2274.

Johnson et al, (1991). Analyt. Biochem. 198, 268-277.

Kennedy et al, (1991). J. Virol. 65,2093-2097.

Laimins (1993). The Biology of Human Papillomaviruses: From Warts to Cancer. Infectious Agents and Disease 2, 74-86.

Lambert (1991). J. Virol. 65, 3417-3420.

Low et al, (1996). J. Mol. Biol. 260(3), 359-368.

Marks et al, (1992). Bio-technology 10(7), 779-783.

McClean et al, (1990) J. Clin. Pathol. 43, 488-492.

Meyers et al, (1992). Science 257, 971-973.

Nissim et al, (1994). EMBO J. 13, 692-698.

Palefsky et al, (1991). J. Clin. Invest. 87, 2132-2141.

Pope et al, (1996). Immunotechnology 2(3), 209-217.

Pray et al, (1995). Virology 206, 679-685.

Roberts & Wientraub (1996). Cell 46, 741-752.

Roberts et al, (1994). J. Virol 68(10), 6432-6455.

Roberts et al, (1993). Virology 197,176-187.

Rogel-Gaillard et al, (1992). J. Virol. 66(2), 816-823.

Rogel-Gaillard et al, (1993). J. Invest. Dermatol. 101, 843-851.

Schier et al, (1996). J. Mol, Biol. 263(4), 551-567.

Schneider (1994). Intervirology 37(3-4), 201-214.

Sherman & Schlegel (1996). J Virol. 70, 3269-3279.

Stoler et al, (1990). J. Virol. 64, 3310-3318.

Stoppler et al, (1996). J. Virol. 70,6987-6993.

Villiers de (1994) Human pathogenic papillomavirus types: an update. In “Human Pathogenic Papillomaviruses” (H. Zur Hausen, Ed). ppl 1-12. Springer-Verlag, New York.

Zheng et al, (1996). J. Virol. 70, 4691-4699.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. A method for detecting abnormalities in a sample from a patient, said method comprising:
(a) obtaining a sample of the patient's cells;
(b) contacting said cells with two or more molecule(s), wherein at least one molecule binds a papilloma virus associated nucleic acid or antigen, and at least one molecule binds a cell proliferation marker or a viral activity marker; and
(c) detecting said binding.

2. A method according to claim 1, wherein the sample comprises cervical cells.

3. A method according to claim 2, wherein the sample comprises cells collected from a cervical smear.

4. A method according to claim 1, wherein the cells are contacted with the two or more molecules simultaneously.

5. A method according to claim 1, wherein the papilloma virus is human papilloma virus.

6. A method according to claim 1, wherein the human papilloma virus comprises one or more types selected from the group consisting of HPV types 16, 18, 33, 35, 45, 51, 56, 58 and 61.

7. A method according to claim 1, wherein at least one molecule is a molecule that binds specifically to a subset of HPV E4 proteins.

8. A method according to claim 1, wherein at least one molecule is a molecule that binds the papilloma virus E4 protein, and binds within a hydrophilic region of the E4. sequence.

9. A method according to claim 1, wherein the cell proliferation marker comprises a polypeptide which is a member of a preinitiation complex of DNA replication.

10. A method according to claim 1, wherein at least one molecule binds a papilloma virus associated nucleic acid or antigen, and at least one molecule binds a viral activity marker.

11. A method according to claim 10, wherein the viral activity marker is p16 or cyclin B.

12. A method according to claim 1, wherein at least one molecule binds a papilloma virus associated nucleic acid or antigen, and at least one molecule binds a cell proliferation marker.

13. A method according to claim 12, wherein the cell proliferation marker comprises one or more polypeptides selected from the group consisting of CDC6, MCM2, MCM3, MCM4, MCM5, MCM6, MCM7, Cdc7 protein kinase, Dbf4, Cdc14 protein phosphates, Cdc45, PCNA, Ki67, KiS1, cyclin A and MCM10.

14. A method according to claim 12, wherein the cell proliferation marker is not an MCM polypeptide.

15. A method according to claim 13, wherein the cell proliferation marker is selected from the group consisting of cyclin A, Ki67 and PCNA.

16. A method according to claim 6, wherein the HPV or marker of cell proliferation or viral activity, or both, are detected at the mRNA stage.

17. A method according to claim 1, wherein the molecule binds a papilloma virus associated nucleic acid or antigen, or binds a marker of cell proliferation or viral activity, is an antibody or an antigen-binding fragment thereof.

18. A method according to claim 1, wherein the cell proliferation marker or marker of viral activity is induced by E6 and/or E7.

19. A method for detecting abnormalities in a sample from a patient, said method comprising:
(a) obtaining a sample of the patient's cells;
(b) contacting said cells with two or more molecule(s), wherein at least one molecule binds one papilloma virus associated antigen or a plurality of papilloma virus associated antigens, and at least one molecule binds one cell proliferation or viral activity marker, or a plurality of cell proliferation or viral activity markers; and
(c) detecting said binding.

20. A kit comprising two or more molecule(s), wherein at least one molecule binds a papilloma virus associated antigen, and at least one molecule binds a marker of cell proliferation or viral activity, and packaging materials therefor.

21. A method for assessing the risk associated with cellular abnormality in a patient sample, comprising obtaining a sample of the patient's cells; contacting said cells with two or more molecule(s), wherein at least one molecule binds a papilloma virus associated nucleic acid or antigen, and at least one molecule binds a cell proliferation marker or a plurality of cell proliferation markers and/or marker(s) of viral activity; and categorising the risk according to (a) the presence of cell proliferation markers, and (b) the detection of high or low risk HPV virus infection.
